(19) United States
(12) Patent Application Publication
(10) Pub. No.: US 2005/0256124 A1
(43) Pub. Date: Nov. 17, 2005

(54) Melanin-concentrating hormone receptor antagonists and compositions and methods related thereto

(75) Inventors: Val Goodfellow, Encinitas, CA (US); Brian P. Dyck, San Diego, CA (US); Liren Zhao, San Diego, CA (US); Junko Tamiya, Oceanside, CA (US); Jonathan Grey, San Diego, CA (US); Warren Wade, San Diego, CA (US); Martin Rowbottom, San Diego, CA (US); Mingzhu Zhang, San Diego, CA (US); Troy D. Vickers, San Diego, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Neurocrine Biosciences, Inc., San Diego, CA (US)

(21) Appl. No.: 11/107,317

(22) Filed: Apr. 15, 2005

Related U.S. Application Data
(60) Provisional application No. 60/562,389, filed on Apr. 15, 2004.

(51) Int. Cl. A61K 31/503; A61K 31/501; A61K 31/519; A61K 31/4745; A61K 31/4439; C07D 49/14; C07D 498/02; C07D 41/14

(52) U.S. Cl. 514/248; 514/252.03; 514/260.1; 514/337; 544/236; 544/238; 544/279; 546/153; 546/141; 546/276.4; 514/309; 514/312

(57) ABSTRACT

Melanin-concentrating hormone (MCH) receptor antagonists are disclosed having utility for the treatment of MCH receptor-based disorders such as obesity. The compounds of this invention have the following structure: 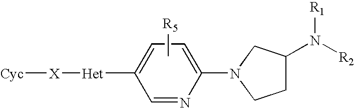
including stereoisomers, prodrugs, and pharmaceutically acceptable salts thereof, wherein R₁, R₂, R₅, Het, X and Cyc are as defined herein. Also disclosed are compositions containing a compound of this invention, as well as methods relating to the use thereof.

### CROSS-REFERENCE TO RELATED APPLICATION

This application claims the benefit of U.S. Provisional Patent Application No. 60/552,389 filed Apr. 15, 2004, which application is incorporated herein by reference in its entirety.

### STATEMENT OF GOVERNMENT INTEREST

Partial funding of the work described herein was provided by the U.S. Government under Grant No. 2R44-DK59107-02 provided by the National Institutes of Health. The U.S. Government may have certain rights in this invention.

### BACKGROUND OF THE INVENTION

1. Field of the Invention

This invention generally relates to antagonists of melanin-concentrating hormone receptors, and to compositions and methods related thereto.

2. Description of the Related Art

Melanin-concentrating hormone (MCH) is a neuropeptide that exerts a powerful effect on food intake and body weight regulation (Broberger & Hokfelt, PHYSIOL. BEHAV. 2001 November-December; 74(4-5): 669-82.) As a result, this neuropeptide, as well as antagonists to its various receptors, has been investigated for use in therapies relating to eating and body weight regulating disorders.

Experiments where MCH was directly injected into lateral ventricles of the brains of rats resulted in increased consumption of food, indicating that MCH has a role in the regulation of body weight (Qu, et al., NATURE 1996 Mar. 21; 380 (6571):243-7.) The orexigenic (appetite-stimulating) activity is believed to result from MCH binding to a melanin-concentrating hormone receptor (MCH-1R) determined to be a 353 amino acid human orphan G-Protein-Coupled Receptor (GPCR) SLC-1 (Chambers et al., NATURE 1999 Jul. 15; 400(6741): 261-5; Saito et al., NATURE 1999 Jul. 15; 4000(6741): 265-9.) Mice deficient in MCH-1R have normal body weights yet are lean and have reduced fat mass; thus, such mice are less susceptible to diet-induced obesity (Marsh et al., PROC. NATL. ACAD. SCI. 2002 Mar. 5; 99 (5): 3240-5.) A second MCH receptor (MCH-2R) has also been identified (Sailer et al., PROC. NATL. ACAD. SCI. 2001 Jun. 19; 98(13): 7564-9; An et al. PROC. NATL. ACAD. SCI. 2001 Jun. 19; 98(13): 7576-81.)

In view of the biological importance of MCH, a number of researchers have reported peptide or small molecule antagonists of MCH receptors. For example, Merck Research Laboratories has reported peptide ligands consisting of the cyclic core of human MCH that activates both MCH-1R and MCH-2R, and a ligand with selectivity for MCH-1R (Bednarek et al., BIOCHEMISTRY 2002; 41(20): 6383-9.) Takeda Chemical Industries (Takeda) has disclosed the use of (−)-N-[6-(dimethylamino)-methyl]-5,6,7,8-tetrahydro-2-naphthalenyl]-4′-fluoro-[1,1′-biphenyl]-4-carboxamide and derivatives thereof as selective MCH-1R inhibitors (Kakekawa et al., EUR J PHAMOCOL 2002 Mar. 8; 438(3); 129-35; WO 01/21577.) Additional Takeda patent publications directed to MCH antagonists include JP 2001226269; WO 01/21169; WO 01/82925; and WO 01/87834. Synaptic Pharmaceutical Corporation has similarly disclosed MCH receptor antagonists (WO 02/06245), as has Neurogen Corporation (WO 02/04433; US 20020052383 A1.)

Accordingly, there remains a need in the art for novel MCH receptor antagonists, including antagonists of MCH-1R and/or MCH-2R, and for compositions and methods related thereto. The present invention fulfils these needs and provides further related advantages.

### BRIEF SUMMARY OF THE INVENTION

In brief, this invention is generally directed to compounds that function as antagonists to one or more melanin-concentrating hormone (MCH) receptor(s), such as MCH-1R and MCH-2R (or both). This invention is also directed to compositions containing one or more of such compounds in combination with one or more pharmaceutically acceptable carriers, as well as to methods for treating conditions or disorders associated with MCH.

In one embodiment, compounds are disclosed that have the following structure (I): 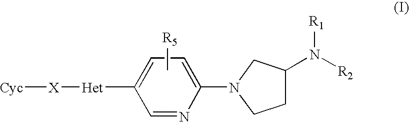
including stereoisomers, prodrugs, and pharmaceutically acceptable salts thereof, wherein R₁, R₂, R₅, Het, X and Cyc are as defined herein.

The compounds of this invention have utility over a broad range of therapeutic applications, and may be used to treat disorders or illnesses, including (but not limited to) eating disorders, body weight disorders, anxiety, depression and CNS disorders. A representative method of treating such a disorder or illness includes administering an effective amount of a compound of this invention, typically in the form of a pharmaceutical composition, to an animal in need thereof (also referred to herein as a “patient,” including a human). Accordingly, and in another embodiment, pharmaceutical compositions are disclosed containing one or more compounds of this invention in combination with a pharmaceutically acceptable carrier.

These and other aspects of this invention will be apparent upon reference to the following detailed description and attached figures. To that end, certain patent and other documents are cited herein to more specifically set forth various aspects of this invention. Each of these documents is hereby incorporated by reference in its entirety.

### DETAILED DESCRIPTION OF THE INVENTION

As mentioned above, the present invention is generally directed to compounds useful as melanin-concentrating hormone (MCH) receptor antagonists. Such compounds have the following structure (I): 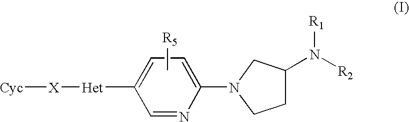
or a stereoisomer, prodrug or pharmaceutically acceptable salt thereof,
wherein:
   R₁ is hydrogen, alkyl, substituted alkyl, aryl, substituted aryl, heterocycle, substituted heterocycle, arylalkyl, substituted arylalkyl, heterocyclealkyl or substituted heterocyclealkyl;
   R₂ is hydrogen, alkyl, substituted alkyl, —C(O)R₃ or —S(O)₂R₃;
   or R₁ and R₂ together with the nitrogen to which they are attached form a heterocycle optionally substituted with 1, 2 or 3 R₄ groups;
   R₃ is hydroxy, alkyl, substituted alkyl, —O(alkyl), —O(substituted alkyl), aryl, substituted aryl, heterocycle, substituted heterocycle, arylalkyl, substituted arylalkyl, heterocyclealkyl or substituted heterocyclealkyl;
   R₄ is halogen, hydroxy, alkyl, substituted alkyl, —O(alkyl), —O(substituted alkyl), aryl, substituted aryl, heterocycle, substituted heterocycle, arylalkyl, substituted arylalkyl, heterocyclealkyl or substituted heterocyclealkyl;
   R₅ is hydrogen, halogen, cyano, alkyl, substituted alkyl, —O(alkyl), —O(substituted alkyl), —C(O)R₃, —S(O)R₆, —S(O)₂R₃, —C(O)N(R₇)₂, —NHC(O)R₇, or —N(R₇)₂;
   R₆ is alkyl, substituted alkyl, —O(alkyl), —O(substituted alkyl), aryl, substituted aryl, heterocycle, substituted heterocycle, arylalkyl, substituted arylalkyl, heterocyclealkyl or substituted heterocyclealkyl;
   R₇ is, at each occurrence, the same or different and independently hydrogen, alkyl or substituted alkyl;
   Het is 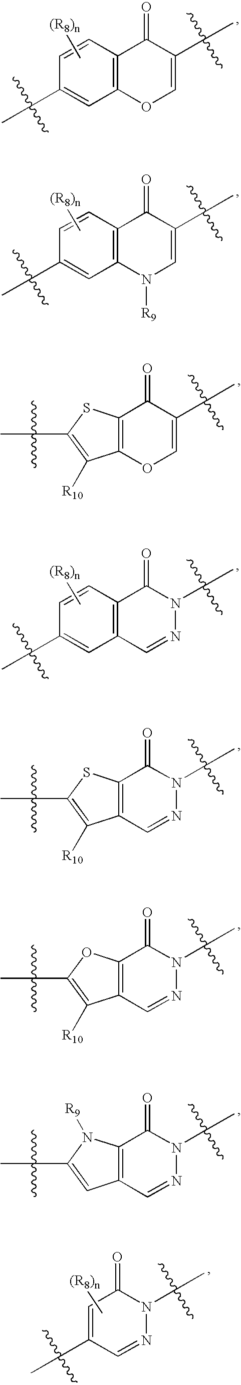 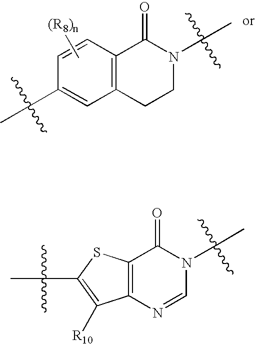
      wherein for each Het the connection to the pyridyl ring of structure (I) is from the atom adjacent to the ketone;
R₈ is, at each occurrence, the same or different and independently halogen, alkyl or substituted alkyl;
R₉ is hydrogen, alkyl or substituted alkyl;
R₁₀ is hydrogen, halogen, alkyl or substituted alkyl;
n is 0, 1 or 2;
X is a bond or —O—; and
Cyc is cycloalkyl, substituted cycloalkyl, aryl, substituted aryl, heterocycle or substituted heterocycle.

As used herein, the above terms have the following meaning:

“Alkyl” means a straight chain or branched, noncyclic or cyclic, unsaturated or saturated a liphatic hydrocarbon containing from 1 to 10 carbon atoms, whle the term “lower alkyl” has the same meaning as alkyl but contains from 1 to 6 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, —CH₂-cyclohexyl, and the like; while unsaturated cyclic alkyls include cyclopentenyl, cyclohexenyl, —CH₂-cyclohexenyl, and the like. Cyclic alkyls are also referred to herein as a “cycloalkyl.” Unsaturated alkyls contain at least one double or triple bond between adjacent carbon atoms (referred to as an “alkenyl” or “alkynyl”, respectively.) Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Aryl” means an aromatic carbocyclic moiety such as phenyl or naphthyl.

“Arylalkyl” means an alkyl having at least one alkyl hydrogen atom replaced with an aryl moiety, such as benzyl (i.e., —CH₂-phenyl), —(CH₂)₂-phenyl, —(CH₂)₃-phenyl, —CH(phenyl)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, triazolyl, tetrazolyl, oxadiazolyl, benzoxadiazolyl, thiadiazolyl, indazolyl and quinazolinyl.

“Heteroarylalkyl” means an alkyl having at least one alkyl hydrogen atom replaced with a heteroaryl moiety, such as —CH₂-pyridinyl, —CH₂-pyrimidinyl, and the like.

“Heterocycle” (also referred to herein as a “heterocyclic ring”) means a 4- to 7-membered monocyclic, or 7- to 10-membered bicyclic, heterocyclic ring which is saturated, unsaturated, or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, piperizinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocyclealkyl” means an alkyl having at least one alkyl hydrogen atom replaced with a heterocycle moiety, such as —CH₂-morpholinyl, —CH₂-pyrrolidinyl, and the like.

The term “substituted” as used herein means any of the above groups (i.e., alkyl, cycloalkyl, aryl, arylalkyl, heteroaryl, heteroarylalkyl, heterocycle and heterocyclealkyl) wherein at least one hydrogen atom is replaced with a substituent. In the case of an oxo substituent (“═O”) two hydrogen atoms are replaced. When substituted, “substituents” within the context of this invention include oxo, halogen, hydroxy, cyano, nitro, amino, alkylamino, dialkylamino, alkyl, alkoxy, thioalkyl, sulfonylalkyl, haloalkyl, hydroxyalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NRₐNR_{b}, —NRₐC(═O)OR_{b} —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ, —S(═O)₂ORₐ, —CH₂S(═O)₂Rₐ, —CH₂S(═O)₂NRₐR_{b}, ═NS(═O)₂Rₐ, —S(═O)₂NRₐR_{b}, wherein Rₐ and R_{b} are the same or different and independently hydrogen, alkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl.

“Halogen” means fluoro, chloro, bromo and iodo.

“Haloalkyl” means an alkyl having at least one hydrogen atom replaced with halogen, such as trifluoromethyl and the like.

“Hydroxyalkyl” means an alkyl substituted with at least one hydroxyl group (i.e, —OH).

“Alkoxy” means an alkyl moiety attached through an oxygen bridge (i.e., —O-alkyl) such as methoxy, ethoxy, and the like.

“Thioalkyl” means an alkyl moiety attached through a sulfur bridge (i.e., —S-alkyl) such as methylthio, ethylthio, and the like.

“Sulfonylalkyl” means an alkyl moiety attached through a sulfonyl bridge (i.e., —SO₂-alkyl) such as methylsulfonyl, ethylsulfonyl, and the like.

“Alkylamino” and “dialkylamino” mean one or two alkyl moieties attached through a nitrogen bridge (i.e., —N-alkyl) such as methylamino, ethylamino, dimethylamino, diethylamino, and the like.

In one embodiment, compounds of this invention have structure (II) when X is a direct bond, and have structure (III) when X is —O—:

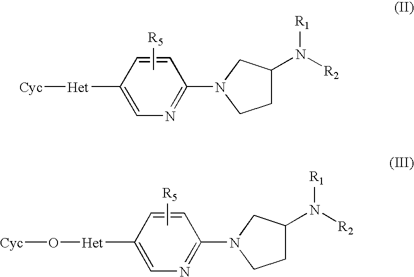

In more specific embodiments of structure (II), compounds of this invention have the following structure (II-1) when Cyc is phenyl, (II-2) when Cyc is N-methylindolyl, and structure (II-3) when Cyc is benzo[1,3]dioxyl:

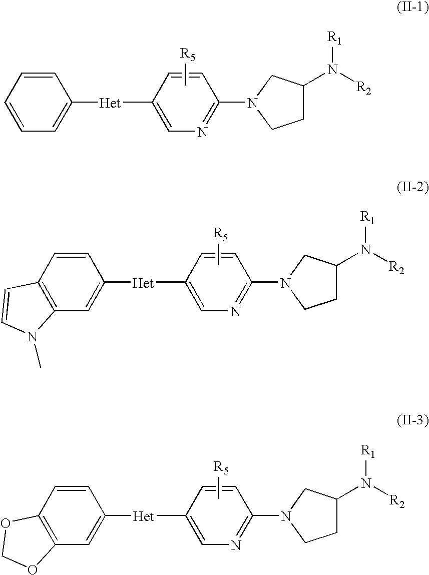

In other more specific embodiments, and depending upon the selection of the Het group, compounds of this invention have one of the following structures (IV) through (XIII):

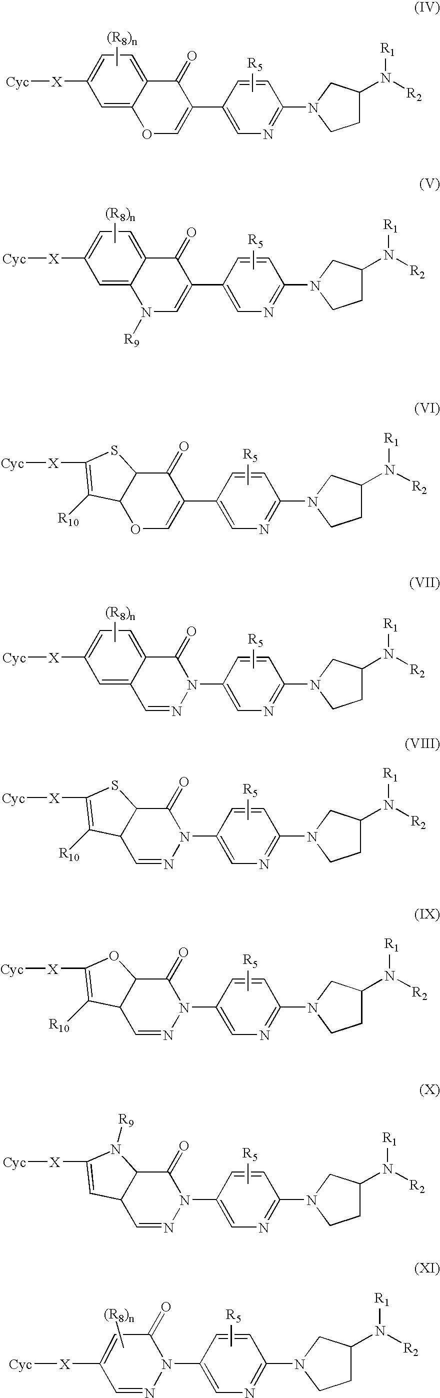

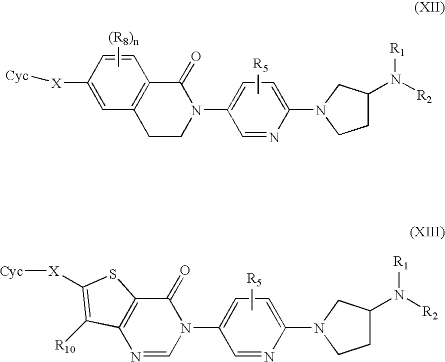

In still other more specific embodiments compounds of this invention have the following structure (XIV) when R₁ and R₂ are both methyl, have structure (XV) when R₁ and R₂ taken together with the nitrogen to which they are attached form pyrrolidine, and have structure (XVI) when R₁ and R₂ taken together with the nitrogen to which they are attached form morpholine:

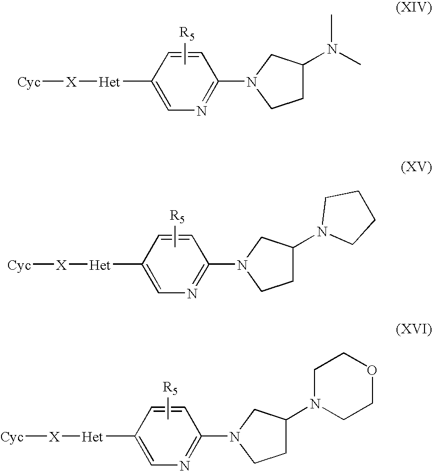

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the compounds of structure (I). Further, in the case of a carboxylic acid (—COOH), esters may be employed, such as methyl esters, ethyl esters, and the like.

With regard to stereoisomers, the compounds of structure (I) may have chiral centers and may occur as racemates, racemic mixtures and as individual enantiomers or diastereomers. Racemic mixtures may be resolved to the pure enantiomeric forms by various procedures known in the art including but not limited to resolution by chromatography. All such isomeric forms are included within the present invention, including mixtures thereof. Compounds of structure (I) may also possess axial chirality that may result in atropisomers. Furthermore, some of the crystalline forms of the compounds of structure (I) may exist as polymorphs, which are included in the present invention. In addition, some of the compounds of structure (I) may also form solvates with water or other organic solvents. Such solvates are similarly included within the scope of this invention.

The compounds of this invention may be prepared by known organic synthesis techniques, including the methods described in more detail in the Examples, as well as by the following general Reaction Schemes 1 and 2 and more specific Reaction Schemes 3 through 11:

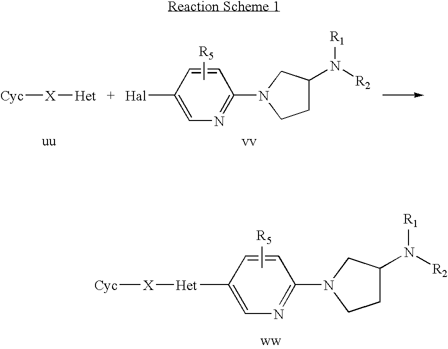

Condensation of reagent uu with halogenated compound vv affords compound ww. This condensation reaction can be realized by methods known in the art including, but not limited to, use of Cs₂CO₃, Cu(I)I and trans-1,2-cyclohexanediamine in an aprotic environment.

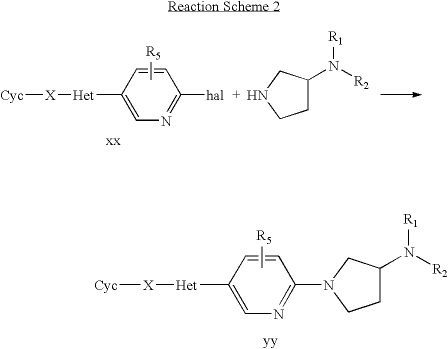

Condensation of halogenated reagent xx with an appropriately substituted aminopyrrolidine affords compound ww. This reaction, well known in the art, can be catalyzed by reagents including, but not limited to, p-toluenesulfonic acid monohydrate.

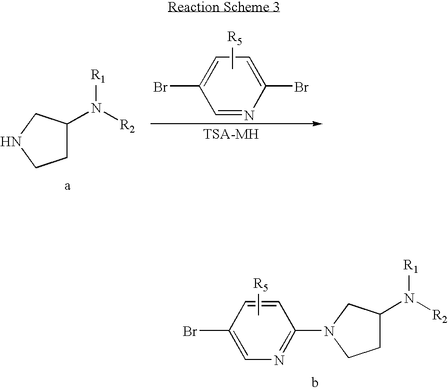

Aminopyrrolidine a, substituted 2,5-dibromopyridine, and p-toluenesulfonic acid monohydrate react to afford after purification pyrimidylpyrrolidylamine b.

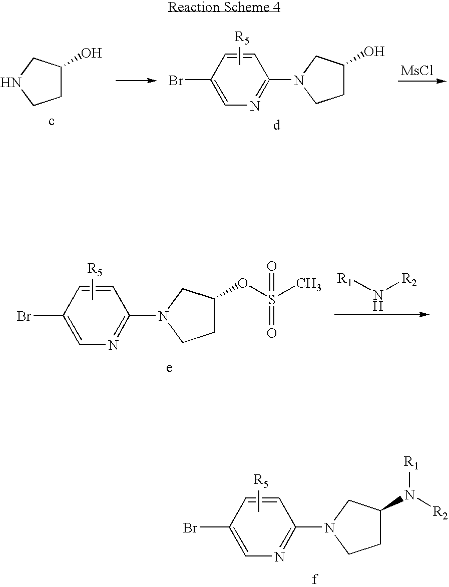

Hydroxypyrrolidine c reacts with substituted 2,5-dibromopyridine and p-toluenesulfonic acid monohydrate by the reaction given in Reaction Scheme 3 to afford pyridinylpyrrolidinol d. Reaction of compound d with methanesulfonyl chloride affords compound e. Heating of mesyl compound e with amine affords pyridinylpyrrolidinylamine f. Compound c as drawn in Reaction Scheme 4 is of the (R) configuration. Starting with the (S) enantiomer of compound c and proceeding with the steps of Reaction Scheme 4 affords enantiomer f′.

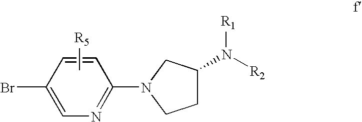

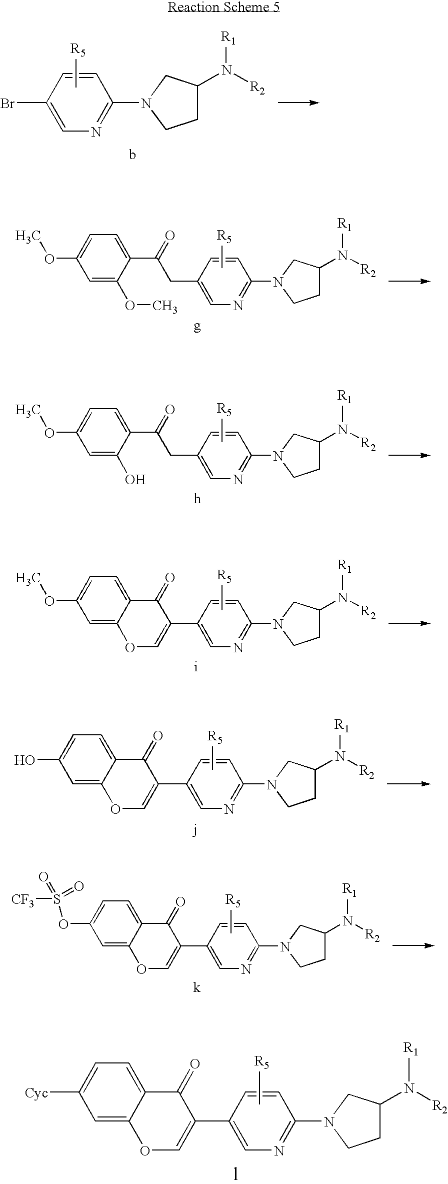

Reaction of sodium t-butoxide, palladium(II) acetate, 2-(dicyclohexylphosphino)-2′-methylbiphenyl, compound b (Reaction Scheme 3) and 2′,4′-dimethoxyacetophenone affords bis-methoxy compound g. Demethylation with boron tribromide affords alcohol h. Compound h reacts with N,N-dimethylformamide dimethyl acetal to afford compound i. Reaction of compound i and sodium iodide in hydrobromic acid affords alcohol j. Reaction of compound j with trifluoromethanesulfonic anhydride affords sulfonate k which undergoes substitution with an arylboronic acid to afford compound l.

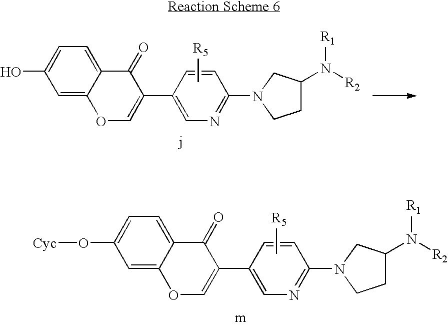

A suspension of compound j (Reaction Scheme 5), arylbromide and Cu(I) oxide reacts with time at elevated temperature to afford after purification compound m.

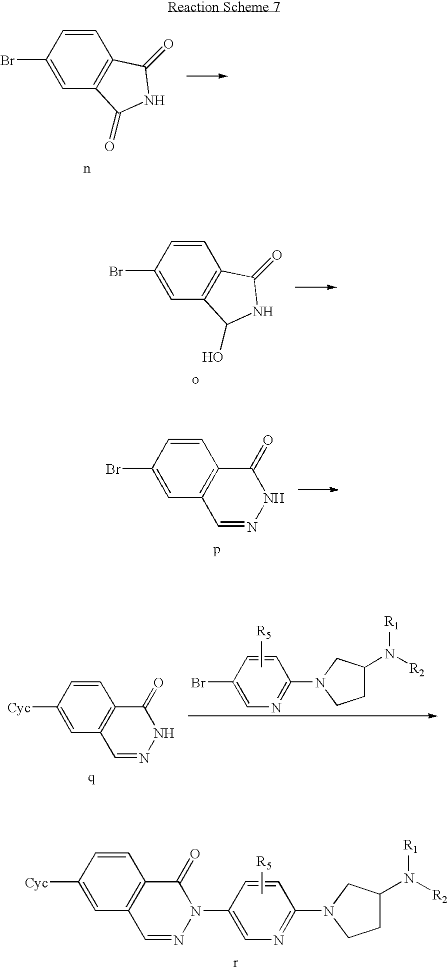

Bromophthalimide n reacts with zinc powder and copper(II) sulfate pentahydrate in a stirred aqueous sodium hydroxide suspension to afford after neutralization and purification compound o. Compound o reacts with hydrazine in water to afford bromophthalazinone p. Substitution at the bromine is achieved by reaction of compound p with arylboronic acid and potassium carbonate in the presence of [1,1′-bis(diphenylphosphino)-ferrocene]dichloropalladium(II), the so-called “Suzuki” reaction (Suzuki, A. J., ORGANOMETALLIC CHEM. 1999, 576, 147-168,) which affords after purification compound q. A suspension of compound q, compound b (Reaction Scheme 3), Cs₂CO₃, Cu(I)I and trans-1,2-cyclohexanediamine in dioxane reacts to afford compound r.

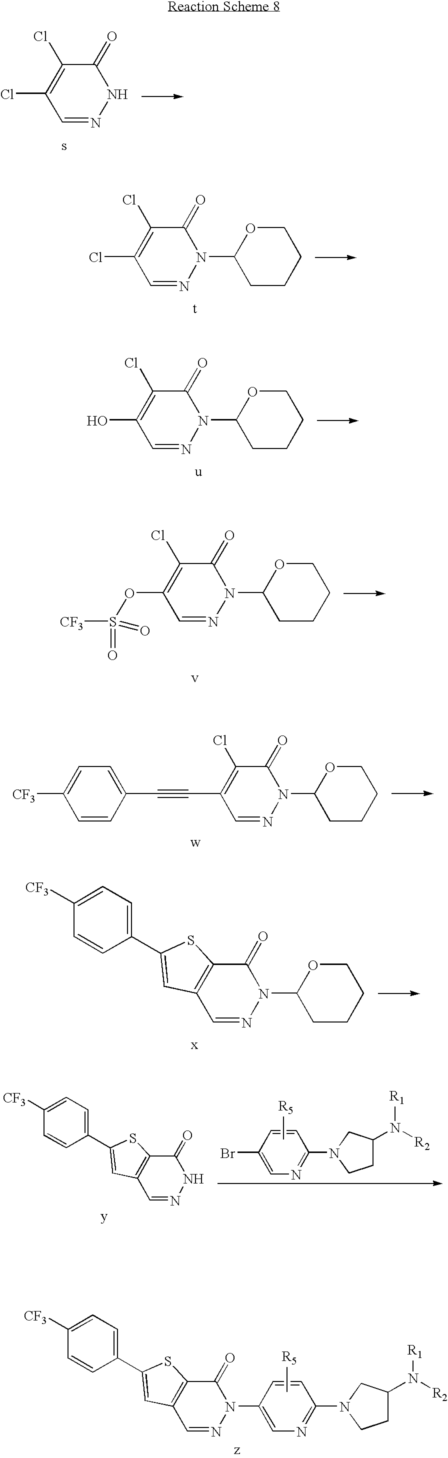

Compound s (4,5-dichloro-2H-pyridazin-3-one) reacts with 3,4-dihydro-2H-pyran and p-toluenesulfonic acid monohydrate in THF suspension to afford after purification compound t. Pyridazone t reacts with KOH in ethylene glycol to afford after purification compound u. Reaction of compound u with trifluoromethanesulfonic anhydride affords compound v. Sulfonate v reacts with dichlorobis (triphenylphosphine) palladium(II), Cu(I)I, tetra-n-butylammonium iodide, TEA, and 4-(trifluoromethyl) phenylacetylene to afford after purification acetylene w. Compound w reacts with sodium sulfide nonahydrate in DMF to afford after purification compound x. Compound x loses dihydropyran when treated with HCl in methanol to afford after purification compound y. Compounds y and b react under the conditions described in Reaction Scheme 7 to afford compound z.

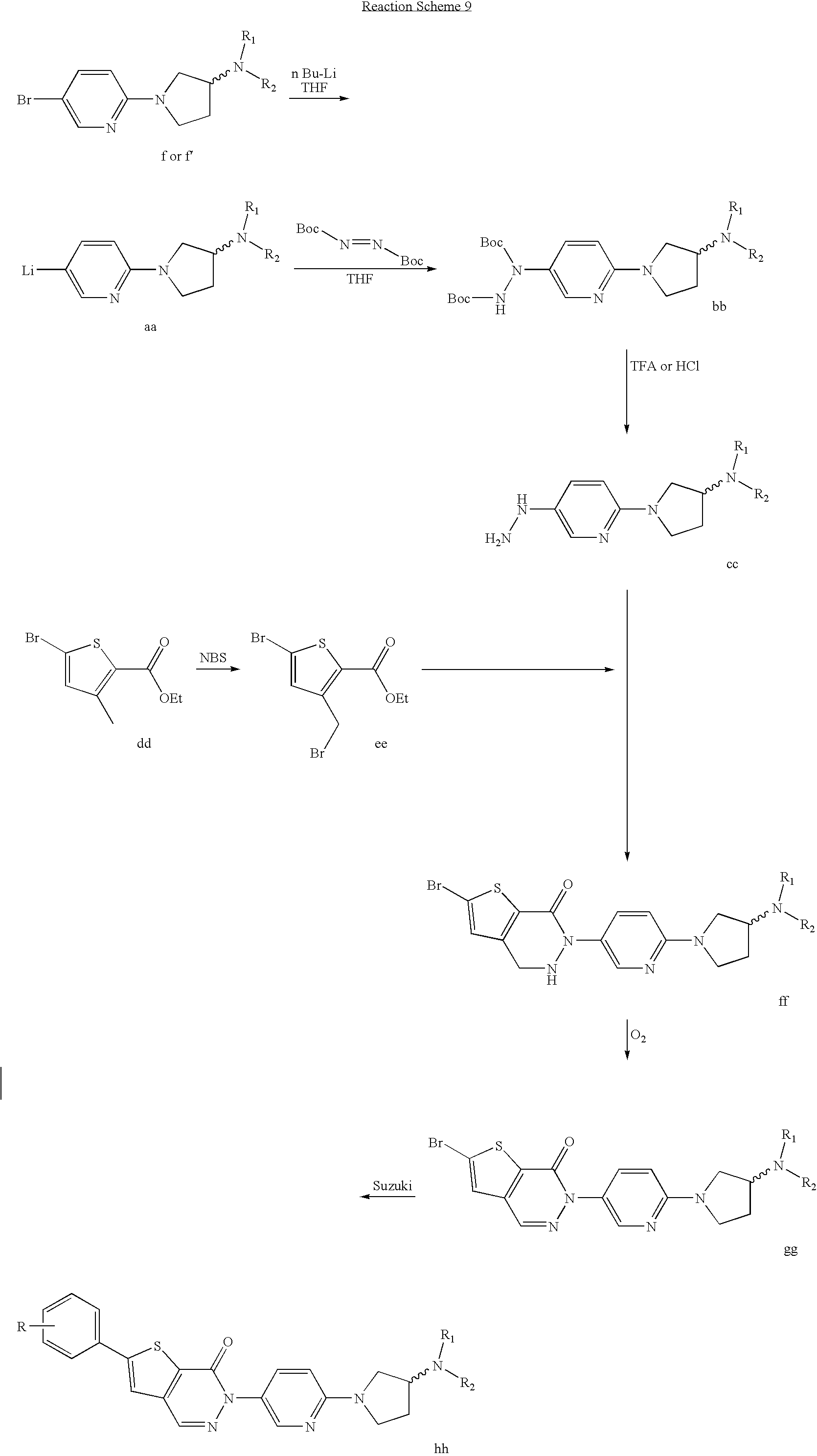

Pyridinylpyrrolidinylamine f or f′ (Reaction Scheme 4) reacts with n-butyllithium in THF to afford lithiated compound aa. Compound aa reacts with di-tert-butyl azodicarboxylate in THF to afford addition compound bb. Removal of the butyloxycarbonyl protecting groups of compound bb in strong acid affords compound cc. 5-Bromo-3-methyl-thiophene-2-carboxylic acid ethyl ester (compound dd) reacts with NBS to afford 5-dibromo-3-bromomethyl-thiophene-2-carboxylic acid ethyl ester (compound ee) which reacts with compound cc to afford dihydrothienopyridazinone ff. Oxidation of compound ff affords the thienopyridazinone gg. Palladium-catalyzed cross-coupling of aryl halide with arylboronic acid in the presence of Pd, a suitable phosphine ligand, and base employing the Suzuki reaction referenced in Reaction Scheme 7 affords compound hh.

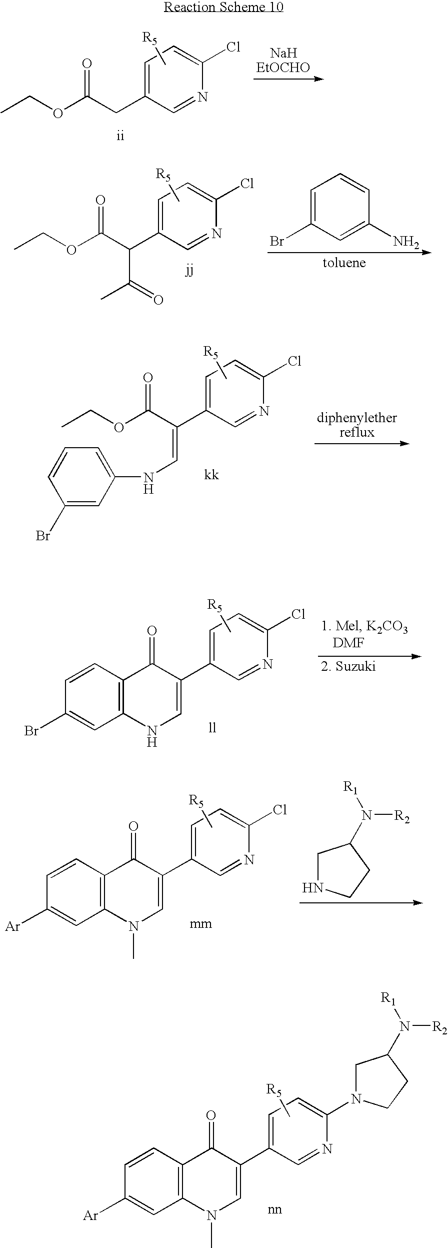

Pyridine ii reacts with NaH and ethylformate to afford compound jj. Compound jj adds 2-bromoaniline upon reflux in toluene to afford compound kk. Compound kk upon reflux in diphenylether loses ethoxide and undergoes ring closure to afford compound ll. Compound ll reacts first with methyliodide and K₂CO₃ in DMF and then under Suzuki conditions to afford quinolone mm. Compound mm reacts with aminopyrrolidine to afford compound nn.

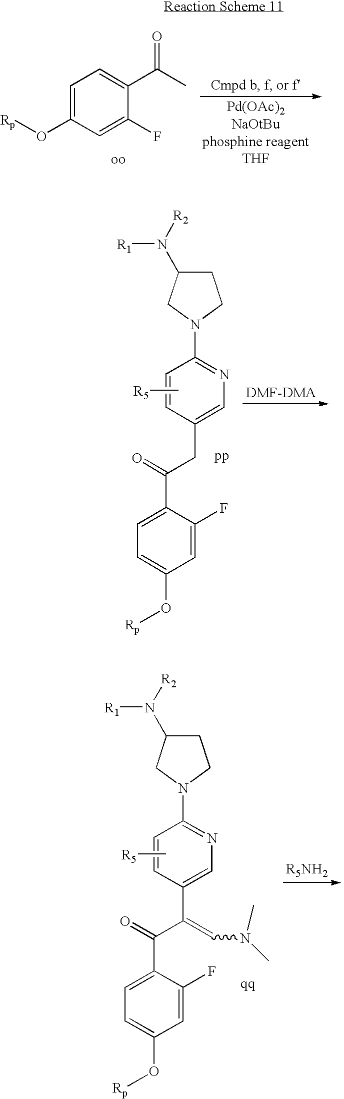

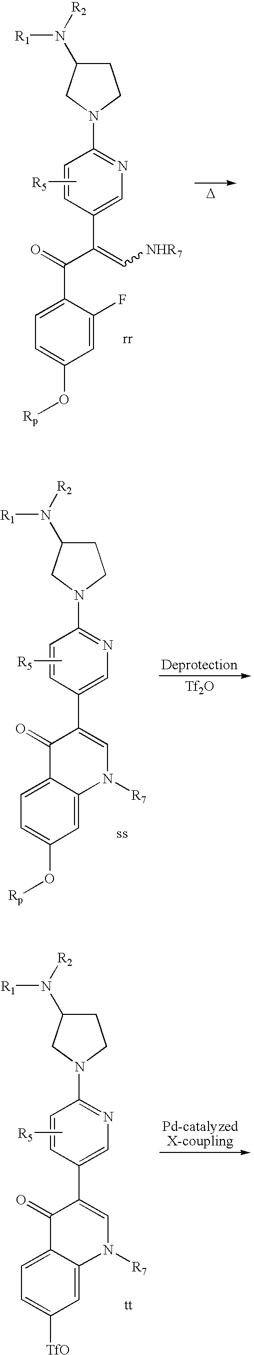

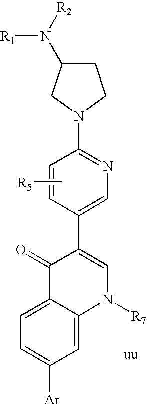

Fluorophenylethanone oo, protected with group Rₚ, adds compound b, f, or f′ in the presence of Pd(OAc)₂, NaOtBu, and 2(dicyclohexylphosphino)2′-methylbiphenyl in THF to afford compound pp. Reaction of compound pp with DMF-DMA affords bis-amino compound qq. The dimethylamino functionality of compound qq is substituted by R₅NH₂ to afford compound rr. With heat, compound rr cyclizes to afford quinolone ss. Deprotection of compound ss by the removal of protecting group Rₚ followed by reaction with triflic anhydride affords triflate tt. Pd-catalyzed cross-coupling of compound tt after the method of Suzuki affords compound uu.

The compounds of this invention may be evaluated for their ability to bind to a MCH receptor by techniques known in the art. For example, a compound may be evaluated for MCH receptor binding by monitoring the displacement of an iodonated peptide ligand, typically human [¹²⁵I]-MCH, from cells expressing individual melanin concentrating hormone receptor subtypes. To this end, whole cells expressing the desired melanin concentrating hormone receptor are subjected to nitrogen cavitation, and the membrane fraction is isolated by differential centrifugation. Stock solutions of test compounds are diluted serially in binding buffer (50 mM HEPES+10 mM MgCl₂+2 mM EGTA) and an equal volume mixed with [¹²⁵I]-MCH (0.2 nM final) diluted in binding buffer. Unlabeled MCH is included as a control. Membranes (5-10 μg total protein) are added to each test compound concentration and incubated for 30 minutes at room temperature. Bound radioligand is captured using GF/C glass fiber filter plates treated with 1% PEI and coated with 1% BSA. Free radioligand is removed by three sequential washes with wash buffer (PBS+0.01% Triton X-100.) Kᵢ values are determined by data analysis using appropriate software, such as GraphPad Prizm, and data are plotted as counts of radiolabeled MCH bound versus the log concentration of test compound.

MCH receptors may couple to various G-proteins in vivo. Functional assays of receptor activation have been defined for the MCH receptors based on their coupling to G_{q} proteins. In response to MCH peptides, the MCH receptors may couple to G_{q} and activate phospholipase C resulting in an increased release of intracellular calcium. Melanin concentrating hormone receptor activity can be measured in HEK293 cells expressing individual melanin concentrating hormone receptors by direct measurement of Ca²⁺ levels. For example, HEK293 cells expressing the desired MCH receptor are seeded into 96-well microtiter Poly-D-Lysine-coated plates at a density of 80,000 cells per well and allowed to adhere overnight with incubation at 37° C. in 5% CO₂. Test compounds are diluted in dilution buffer (HBSS+20 mM HEPES+0.1% BSA+2.5 mM Probenecid) and assessed for antagonist activity over a range of concentrations along with a control agonist MCH. Prior to the assay, cells are loaded with the calcium sensitive dye Fluo-4 for 1 hour at 37° C. Cells are then washed three times with assay buffer (dilution buffer without BSA), and brought to a final volume of 150 μl/well in assay buffer. At the time of assay, 50 μl of test compound is added to each well and allowed to incubate for 2 minutes at room temperature. MCH agonist peptide at a concentration of 10 nM is then added, and intracellular calcium release is measured in real-time using a fluorimetric imaging plate reader (FLIPR.) EC₅₀ values are determined by data analysis using appropriate software such as GraphPad Prizin, and data are plotted as relative fluorescent units produced versus log concentration of compound.

As mentioned above, the compounds of this invention function as antagonists to the MCH receptor 1, and are thereby useful in the treatment of a variety of conditions or diseases including (but not limited to) eating disorders and obesity. The compounds of the present invention may also be used in combination therapy with agents that modify food intake or appetite, and are also included within the scope of this invention. Such agents include, but are not limited to, other MCH receptor ligands, or ligands of the leptin, NPY, melanocortin, serotonin or B₃ adrenergic receptors.

In another embodiment, compounds of this invention may be useful as anti-anxiety and/or anti-depression agents through interaction with the MCH receptor. These compounds may also be used in combination therapy with other anti-anxiety agents or anti-psychotics for the treatment of anxiety, depression, schizophrenia, and other CNS diseases.

In a further embodiment, compounds of this invention may be useful to treat digestive disorders and to modify fertility and sexual function through interaction with the MCH receptor in humans and other mammals. By using PCR of reverse-transcribed RNA, low levels of MCH gene transcripts were detected in testis, stomach, and intestine of Sprague-Dawley and Wistar rats. (Hervieu, NEUROENDOCRINOLOGY 1995 April; 61(4):348-64). In testis, the MCH transcripts and pro-MCH-derived peptide immunoreactivities were found at the periphery of the seminiferous tubules, suggesting expression in Sertoli cells. In the gastrointestinal (01) tract, the cells expressing MCH RNA species and pro-MCH-derived peptides were predominantly expressed in the antral portion of the stomach and duodenum. The actual cellular location of expression suggests that MCH and associated peptides may play a role in spermatogenesis and in digestive processes. Further studies demonstrated effect of MCH peptide on water and electrolyte secretions at different levels of the GI tract by using the in situ ligated loop technique. (Hervieu, ENDOCRINOLOGY 1996 February; 137(2):561-71). MCH stimulated water, Na, and K fluxes at the proximal colon level and increased Na and K fluxes in the duodenum. MCH also increased bicarbonate absorption in the jejunum. Direct administration of MCH to ventromedial nucleus (VMN) and medial preoptic area (MPOA) in female rats has been reported to initiate sexual activity (Gonzales et al., PEPTIDES 1996 17(1):171-7). Further studies suggested that MCH has a stimulatory effect on LH release (Gonzales et al., NEUROENDOCRINOLOGY 1997 October; 66(4):254-62; Murray J., NEUROENDOCRINOL 2000 November; 12(11):1133-9). MCH has also been shown to be involved in release of other gonadotropins (Chiocchio, BIOL REPROD. 2001 May; 64(5):1466-72). Thus antagonists of MCH may be useful in the development of agents to treat digestive disorders of the stomach and colon and may have a role in modulating fertility and sexual function.

In a further embodiment, compounds of this invention may be useful in treating urinary disorders. In studies of the cardiovascular and metabolic actions of intracerebroventricular (i.c.v.) infusion of MCH, and the pro-MCH derived peptide Neuropeptide-E-I (NEI), in conscious, chronically instrumented sheep, the i.c.v. infusion of MCH or NEI is shown to be capable of producing diuretic, natriuretic and kaliuretic changes in conscious sheep, triggered by a possible increase in plasma volume as indicated by the changes in hematocrit (Parkes, J NEUROENDOCRINOL. 1996 January; 8(1):57-63). These results, together with anatomical data reporting the presence of MCH/NEI in fluid regulatory areas of the brain, indicate that MCH/NEI may be an important peptide involved in the central control of fluid homeostasis in mammals. Hence, antagonists of MCH such as the compounds of the present invention may be used to treat urinary disorders including urinary incontinence, overactive bladder and urge urinary incontinence.

In a further embodiment, compounds of this invention may be useful in treating disorders of the immune system including autoimmune diseases and inflammatory diseases. Studies suggest that MCH peptide and MCHR-1 are expressed both in rodent and human immune cells. Further evidence shows expression of MCH increases with activation of T-cells indicating MCH antagonists may be useful in treating diseases associated with immune response including auto-immune diseases and inflammation.

The following methods can be used to evaluate the effect of the treatment of obesity and anxiety in animal test objects:

Deprivation-Induced Feeding

In this acute model, the suppression of deprivation-induced food intake during the light cycle is examined. Male Sprague-Dawley rats are habituated to a palatable diet (Research Diets D12266B) over 3 days prior to testing. Rats are food deprived for 23 hours before the test. On test day, animals are moved to a testing room, the drug is administered, and food intake is measured hourly up to 6 hours. Vehicle and 3 doses of drug are administered to separate groups of animals (n=8 per group). A two-way (time×dose) analysis of variance with Bonferroni post-hoc comparison is used to determine significant treatment effects.

Effects of Chronic Drug Administration in Diet-Induced Obese Rats

To induce obesity, male Sprague-Dawley rats are fed a medium high fat (32%) diet (Research Diets D12266B) for approximately 12 weeks prior to experimentation. Before drug administration begins, animals are habituated to handling and the oral dosing procedure for 1 week. During this period, food intake (corrected for spillage) and body weight are measured daily. Animals are subsequently divided into groups (n=10 per group), balanced for body weight and food intake. Groups consist of a vehicle control, a positive control (e.g., fenfluramine), and one of 3 drug doses. Treatments are then given orally once or twice daily over 4 weeks. Food intake and body weight are measured daily. At the end of dosing, animals are sacrificed and blood is taken to determine plasma levels of glucose, insulin, leptin, free fatty acids, and corticosterone. Gastrocnemius muscle, inguinal fat pads, and retroperitoneal fat pads are dissected and weighed. Dependent measures are analyzed using analysis of variance and Bonferroni post-hoc comparisons.

Guinea Pig Pup Ultrasonic Vocalization

Separation of guinea pig pups from their mothers and littermates elicits distress vocalizations. Studies have indicated that this behavioral response is sensitive to anxiolytic drugs. In this model of anxiety, guinea pig pups (5-26 days of age) are separated from their mothers and littermates and placed into a circular open field of 45 cm in diameter. The floor is divided into sections with painted lines so that locomotor activity as well as vocalizations can be monitored. A microphone is situated above the open field and connected to an Ultravox system (Noldus, Wageningen); the number of vocalizations emitted by each animal is then counted. Prior to testing, pups are screened for vocalizations. Pups that make fewer than 200 vocalizations during a 5 min isolation test are excluded from the study. Pups fulfilling this criterion are subsequently tested during five sequential tests of 5 minutes each, with 3-4 washout days between each test. Each pup receives vehicle, the positive reference compound and 3 doses of drug in a randomized, balanced design. Analysis of variance is used to determine differences among treatment conditions.

In another embodiment, pharmaceutical compositions containing one or more compounds of this invention are disclosed. For the purposes of administration, the compounds of the present invention may be formulated as pharmaceutical compositions. Pharmaceutical compositions of the present invention comprise a compound of structure (I) and a pharmaceutically acceptable carrier and/or diluent. The compound is present in the composition in an amount that is effective to treat a particular disorder of interest, and preferably with acceptable toxicity to the patient. Typically, the pharmaceutical composition may include a compound of this invention in an amount ranging from 0.1 mg to 250 mg per dosage depending upon the route of administration, and more typically from 1 mg to 60 mg. One skilled in the art can readily determine appropriate concentrations and dosages.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets that contain, in addition to a compound of this invention, dispersing and surface-active agents, binders, and lubricants. One skilled in this art may further formulate the compound in an appropriate manner, and in accordance with accepted practices, such as those disclosed in REMINGTON'S PHARMACEUTICAL SCIENCES, Gennaro, Ed., Mack Publishing Co., Easton, Pa. 1990.

In another embodiment, the present invention provides a method for treating a condition related to an MCH receptor. Such methods include administration of a compound of the present invention to a warm-blooded animal in an amount sufficient to treat the condition. In this context, “treat” includes prophylactic administration. Such methods include systemic administration of compound of this invention, preferably in the form of a pharmaceutical composition as discussed above. As used herein, systemic administration includes oral and parenteral methods of administration. For oral administration, suitable pharmaceutical compositions include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parental administration, the compounds of the present invention can be prepared in aqueous injection solutions that may contain buffers, antioxidants, bacteriostats, and other additives commonly employed in such solutions.

The following examples are provided for purposes of illustration and not for purposes of limitation.

### EXAMPLES

Analytical HPLC-MS Method 1

Platform: Agilent 1100 series: equipped with an auto-sampler, an UV detector (220 nM and 254 nM), a MS detector (APCI);

HPLC column: YMC ODS AQ, S-5, 5μ, 2.0×50 mm cartridge;

HPLC gradient: 1.0 mL/minute, from 10% acetonitrile in water to 90% acetonitrile in water in 2.5 minutes, maintaining 90% for 1 minute. Both acetonitrile and water have 0.025% TFA.

Analytical HPLC-MS Method 2

Platform: Agilent 1100 series: equipped with an auto-sampler, an UV detector (220 nM and 254 nM), a MS detector (APCI);

HPLC column: Phenomenex Synergi-Max RP, 2.0×50 mm column;

HPLC gradient: 1.0 mL/minute, from 5% acetonitrile in water to 95% acetonitrile in water in 13.5 minutes, maintaining 95% for 2 minute. Both acetonitrile and water have 0.025% TFA.

Analytical HPLC-MS Method 3

Platform: Agilent 1100 series: equipped with an auto-sampler, an UV detector (220 nM and 254 nM), a MS detector (electrospray);

HPLC column: XTerra MS, C₁₈, 5μ, 3.0×250 mm column;

HPLC gradient: 1.0 mL/minute, from 10% acetonitrile in water to 90% acetonitrile in water in 46 minutes, jump to 99% acetonitrile and maintain 99% acetonitrile for 8.04 minutes. Both acetonitrile and water have 0.025% TFA.

Analytical HPLC-MS Method 4

Platform: Agilent 1100 series: equipped with an auto-sampler, an UV detector (220 nM and 254 nM), a MS detector (APCI) and Berger FCM 1200 CO₂ pump module;

HPLC column: Berger Pyridine, PYR 60A, 6μ, 4.6×150 mm column;

HPLC gradient: 4.0 mL/minute, 120 bar; from 10% methanol in supercritical CO₂ to 60% methanol in supercritical CO₂ in 1.67 minutes, maintaining 60% for 1 minute. Methanol has 1.5% water. Backpressure regulated at 140 bar.

Preparative HPLC-MS

Gilson HPLC-MS equipped with Gilson 215 auto-sampler/fraction collector, an UV detector and a ThermoFinnigan AQA Single QUAD Mass detector (electrospray);

HPLC column: BHK ODS-O/B, 5μ, 30×75 mm

HPLC gradients: 35 mL/minute, 10% acetonitrile in water to 100% acetonitrile in 7 minutes, maintaining 100% acetonitrile for 3 minutes.

Preparative HPLC-MS

Platform: Shimadzu HPLC equipped with a Gilson 215 auto-sampler/fraction collector, UV detector and a PE Sciex API150EX mass detector;

HPLC column: BHK ODS-O/B, 5μ, 30×75 mm

HPLC gradient: 35 mL/minute, 10% acetonitrile in water to 100% acetonitrile in 7 minutes, maintaining 100% acetonitrile for 3 minutes, with 0.025% TFA.

Abbreviations:

DCM: dichloromethane

DMF: dimethylformamide

DMF-DMA: N,N-Dimethylformamide Dimethylacetal

DMSO: dimethylsulfoxide

NaBH(OAc)₃: Sodium Triacetoxyborohydride

NMP: 1-Methyl-2-pyrrolidinone

Pd-C: Palladium (10%) on Carbon

TFA: Trifluoroacetic acid

THF: Tetrahydrofuran

t_{R}: retention time (in minutes)

### Example 1

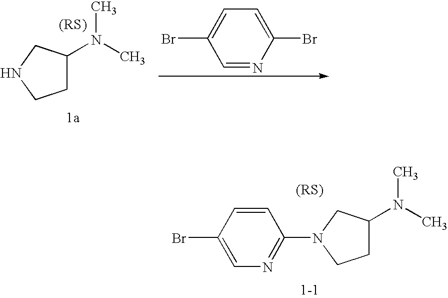 Step 1A:

A mixture of 2,5-dibromopyridine (5.0 g, 21 mmol), 1a (6.0 g, 53 mmol), and p-toluenesulfonic acid monohydrate (1.0 g, 5.3 mmol) was heated in a sealed tube at 140° C. for 14 hours. After cooling to room temperature, the reaction mixture was diluted with 125 mL of DCM. The solution was washed with saturated sodium bicarbonate, brine, dried with MgSO₄, and then concentrated in vacuo to obtain a brown oil. Purification of the crude material by flash column chromatography (elution with 5% methanol and 0.5% aqueous ammonia in DCM) afforded 5.45 g (96%) of compound 1-1, LC-MS 270 (MH⁺.)

Using the appropriate starting materials, the following compounds were prepared according to the above procedures.

| No. | Structure | MW | MH+ |
|---|---|---|---|
| 1-1 | 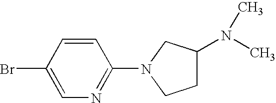 | 270.17 | 270 |
| 1-2 | 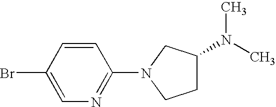 | 270.17 | 270 |
| 1-3 | 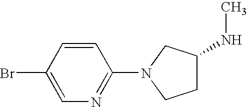 | 256.15 | 256 |
| 1-4 | 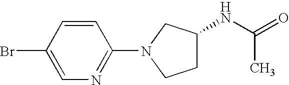 | 284.16 | 284 |
| 1-5 | 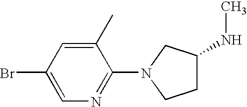 | 270.17 | 270 |
| 1-6 | 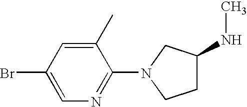 | 270.17 | 270 |
| 1-7 | 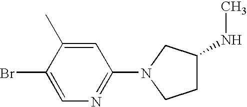 | 270.17 | 270 |

### Example 2

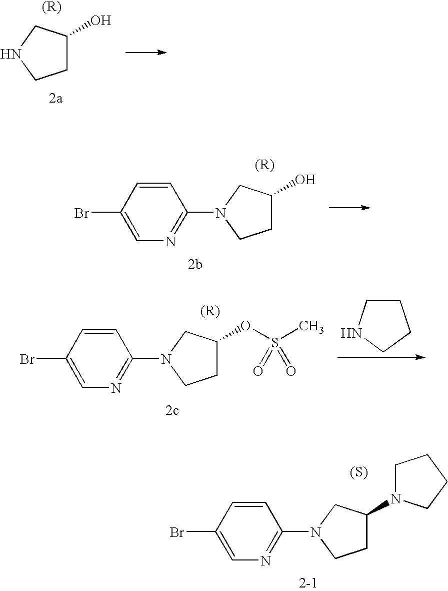 Step 2A:

Compound 2b was prepared from 2a using the procedure as outlined in Step 1A of Example 1, LC-MS 243 (MH⁺.)

Step 2B:

Compound 2b (3.6 g, 15 mmol) was dissolved in 70 mL of DCM with TEA (4.1 mL, 30 mmol) at room temperature. After the mixture was cooled to 0° C. (ice-bath), it was treated with methanesulfonyl chloride (17.3 mL, 22.3 mmol.) After 10 minutes, the ice-bath was removed and the solution was warmed to room temperature and stirring was continued for 1 h. The mixture was diluted with 100 mL of DCM, washed with saturated sodium bicarbonate solution and brine, dried with MgSO₄, and concentrated in vacuo to afford 4.56 g (96%) of 2c as a yellow solid, LC-MS 321 (MH⁺.)

Step 2C:

A solution of 2c (0.56 g, 1.8 mmol) and pyrrolidine (0.64 g, 9.0 mmol) in 6 mL THF was heated at 75° C. in a sealed tube for 14 h. The mixture was cooled to room temperature, filtered, and the THF solution was diluted with 8 mL of saturated sodium bicarbonate solution. The resulting mixture was extracted three times with DCM-IPA (3:1.) The organic extracts were washed with water, dried with MgSO₄, and then concentrated in vacuo to afford a yellow solid. Purification of the crude material by flash column chromatography (elution with 5% methanol and 0.5% aqueous ammonia in DCM) afforded 0.44 g (86%) of 2-1 as a white solid, LC-MS 296 (MH⁺.)

Using the appropriate starting materials, the following compounds were prepared according to the above procedures.

| No. | Structure | MW | MH+ |
|---|---|---|---|
| 2-1 | 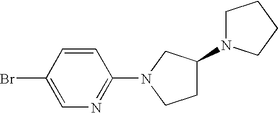 | 296.21 | 296 |
| 2-2 | 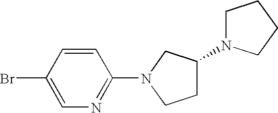 | 296.21 | 296 |
| 2-3 | 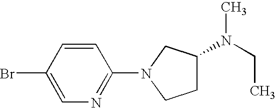 | 284.20 | 284 |
| 2-4 | 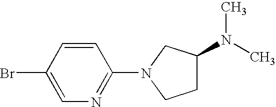 | 270.17 | 270 |
| 2-5 | 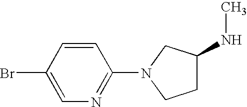 | 256.15 | 256 |
| 2-6 | 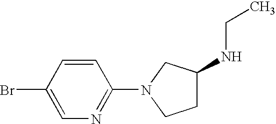 | 270.17 | 270 |
| 2-7 | 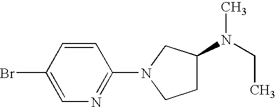 | 284.20 | 284 |
| 2-8 | 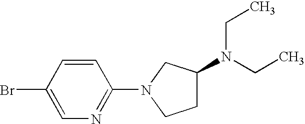 | 298.23 | 296 |

### Example 3

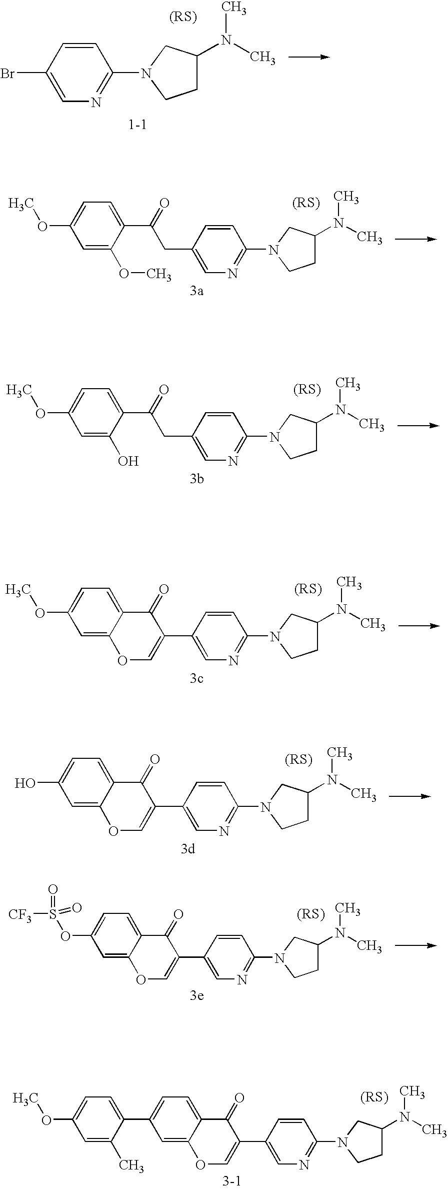 Step 3A:

Nitrogen was bubbled through a stirred suspension of sodium t-butoxide (2.20 g, 22.5 mmol), palladium(II) acetate (0.10 g, 0.45 mmol) and 2-(dicyclohexyl phosphino)-2′-methylbiphenyl (0.33 g, 0.90 mmol) in 50 mL of THF for 10 minutes. Compound 1-1 (2.4 g, 9.0 mmol) and 2′,4′-dimethoxyacetophenone (3.2 g, 18 mmol) were added, and the mixture was heated in a sealed tube at 75° C. for 15 hours. The mixture was diluted with 50 mL of water and then extracted with DCM-IPA (3:1.) The extract was subsequently washed with brine, dried with MgSO₄, and concentrated to afford a brown oil. Purification of the crude material by flash column chromatography (elution with 5% methanol and 0.5% aqueous ammonia in DCM) afforded 3.0 g (91%) of 3a as a red liquid, LC-MS 370 (MH⁺.)

Step 3B:

Boron tribromide in DCM (1.0 M, 17.1 mL, 17.1 mmol) was added dropwise to a solution of 3a (3.0 g, 8.1 mmol) in 10 mL of DCM at −25° C. The reaction mixture was stirred for 15 h, during which time it was gradually warmed to room temperature. The residue was dissolved in 30 mL of methanol and concentrated in vacuo to remove the solvent. After dissolution in methanol and evaporation was repeated, 50 mL of saturated sodium bicarbonate solution was added to the reaction mixture which was then extracted twice with ethyl acetate. The combined organic extracts were washed with brine and dried with MgSO₄ to afford 2.1 g (73%) of 3b as a red oil which solidified on standing, LC-MS 356 (MH⁺.)

Step 3C:

A solution of 3b (1.4 g, 4.2 mmol) and N,N-dimethylformamide dimethyl acetal (5.6 mL, 42 mmol) in 20 mL DCM was stirred at room temperature for 15 h and then concentrated in vacuo to obtain a brown oil. Purification of the crude material by flash column chromatography (elution with 5% methanol and 0.5% aqueous ammonia in DCM) afforded 0.88 g (59%) of 3c as a yellow solid, LC-MS 366 (MH⁺.)

Step 3D:

A suspension of 3c (0.75 g, 2.1 mmol) and sodium iodide (0.46 g, 3.1 mmol) in 25 mL of 48% hydrobromic acid was heated at 100° C. for 24 hours. The reaction mixture was concentrated in vacuo and the residue was neutralized with saturated sodium bicarbonate solution to pH 7. The resulting precipitate was filtered and recrystallized from methanol to afford 0.57 g (78%) of 3d as a red-brown solid, LC-MS 352 (MH⁺.)

Step 3E:

Triethylamine (0.17 g, 1.7 mmol) and 3d (0.15 g, 0.43 mmol) were dissolved in 6 mL of DCM. After trifluoromethanesulfonic anhydride (0.18 g, 0.65 mmol) was added to the solution at 0° C., the mixture was stirred for 30 minutes while it was warmed to room temperature. The mixture was washed with 5% sodium carbonate solution, dried with MgSO₄, and then concentrated in vacuo to afford 0.20 g (97%) of 3e as a yellow solid, LC-MS 484 (MH⁺.)

Step 3F:

Sulfonate 3e (30 mg, 0.062 mmol), 2-methyl-4-methoxybenzeneboronic acid (12 mg, 0.074 mmol), and potassium carbonate (17 mg, 0.12 mmol) were combined in DMF (1 mL) and water (0.1 mL), and nitrogen was bubbled through the mixture for five 5 minutes. The mixture was treated with 1,1′-bis(diphenylphosphino)-ferrocene palladium(II) dichloride DCM complex (5 mg, 0.0006 mmol) and heated at 80° C. for 15 hours. The reaction mixture was filtered and purified by preparative HPLC to afford 10 mg of 3-1 as the trifluoroacetic acid salt, LC-MS 479 (MH⁺.)

Using the appropriate starting materials, the following compounds were prepared according to the above procedures.

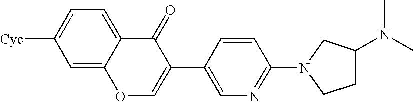

| 1 No. | Cyc | MW | MW⁺ | t_{R} |
|---|---|---|---|---|
| 3-1 | 2-methyl-4-methoxy-phenyl | 455.56 | 456 | 4.596 |
| 3-2 | 4-methoxy-phenyl | 441.53 | 441 | 4.523 |
| 3-3 | 4-Cl-phenyl | 445.95 | 446 | 5.142 |
| 3-4 | 4-CF₃-phenyl | 479.50 | 480 | 5.367 |
| 3-5 | 2-ethyl-phenyl | 439.56 | 440 | 4.619 |
| 3-6 | 6-methoxy-pyridin-3-yl | 442.52 | 443 | 4.099 |
| 3-7 | 6-CF₃-pyridin-3-yl | 480.49 | 481 | 4.221 |
| 3-8 | 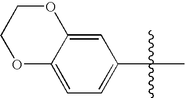 | 469.54 | 470 | 4.281 |
| 3-9 | 4-ethyl-phenyl | 439.56 | 440 | 1.928 |
| 3-10 | 2,4-dichloro-phenyl | 480.39 | 480 | 1.983 |
| 3-11 | 2-chloro-4-methyl-phenyl | 459.97 | 460 | 1.914 |
| 3-12 | 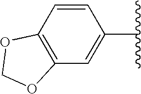 | 455.51 | 456 | 4.356 |
| 3-13 | 2,3-Dihydro-benzofuran-5-yl | 453.54 | 454 | 4.199 |
| 3-14 | 4-Br-phenyl | 490.40 | 490 | 2.086 |
| 3-15 | 3-chloro-4-methoxy-phenyl | 475.97 | 476 | 4.544 |
| 3-16 | 4-trifluoromethoxy-phenyl | 495.50 | 496 | 4.695 |
| 3-17 | 4-ethoxy-phenyl | 455.56 | 456 | 2.014 |
| 3-18 | 1-methyl-1H-indol-6-yl | 464.57 | 465 | 4.488 |
| 3-19 | Benzo[b]thiophen-2-yl | 467.59 | 468 | 2.118 |
| 3-20 | 2,4-dimethoxy-phenyl | 471.55 | 472 | 4.29 1 |
| 3-21 | 4-Cl-2-methy-lphenyl | 459.97 | 460 | 4.794 |
| 3-22 | 4-Cl-3-methyl-phenyl | 459.97 | 460 | 4.793 |
| 3-23 | 4-methoxy-3-methyl-phenyl | 455.56 | 456 | 4.511 |
| 3-24 | 3-F-4-methoxy-phenyl | 459.52 | 460 | 4.498 |
| 3-25 | 4-methyl-phenyl | 425.53 | 426 | 4.104 |
| 3-26 | phenyl | 411.50 | 412 | 4.208 |
| 3-27 | 4-F-phenyl | 429.49 | 430 | 4.158 |
| 3-28 | 2-methyl-4-(pyrazol-1-yl)-phenyl | 491.59 | 492 | 2.137 |

### Example 4

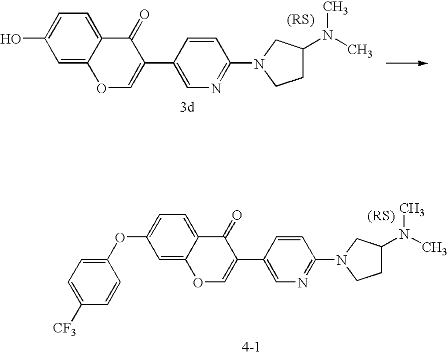 Step 4A:

A suspension of 3d (18 mg, 0.050 mmol), 4-bromobenzotrifluoride (17 mg, 0.075 mmol) and copper(I) oxide (7 mg, 0.05 mmol) in 1 mL of pyridine was heated while stirring at 130° C. for 48 hours. After filtering, the reaction solution was purified by preparative HPLC to afford 10 mg of compound 4-1 as the trifluoroacetic acid salt, LC-MS 495 (MH⁺.)

Using the appropriate starting materials, the following compounds were prepared according to the above procedures.

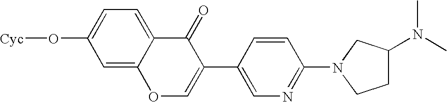

| No. | Cyc | MW | MW⁺ | t_{R} |
|---|---|---|---|---|
| 4-1 | 4-trifluoromethylphenyl | 495.50 | 495 | 4.834 |
| 4-2 | 4-methoxyphenyl | 457.52 | 457 | 4.540 |
| 4-3 | 4-phenoxyphenyl | 519.60 | 520 | 6.123 |
| 4-4 | 4-Cl-phenyl | 461.95 | 462 | 5.308 |
| 4-5 | 4-methyl-phenyl | 441.53 | 442 | 5.234 |
| 4-6 | 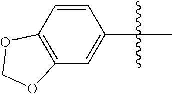 | 469.54 | 470 | 4.829 |
| 4-7 | phenyl | 427.50 | 428 | 4.187 |
| 4-8 | 4-F-phenyl | 445.49 | 446 | 4.438 |
| 4-9 | 4-ethoxy-phenyl | 471.55 | 472 | 1.607 |

### Example 5

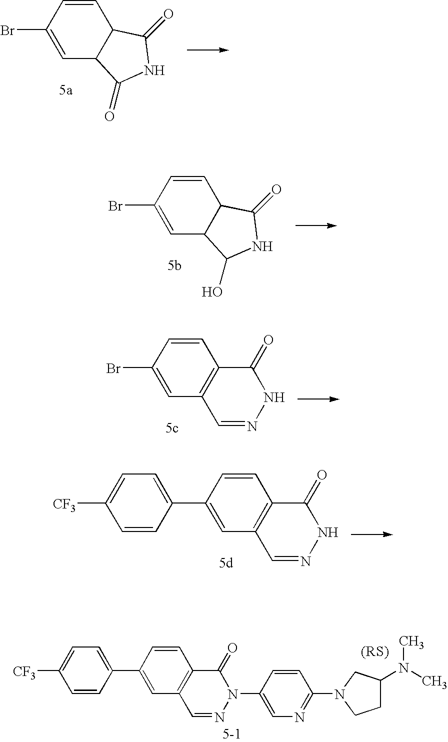 Step 5A:

Bromophthalimide 5a (13.0 g, 57.8 mmol) was added in six portions over 30 minutes to a stirred suspension of zinc powder (4.50 g, 69.2 mmol) and copper(II) sulfate pentahydrate (0.060 g, 0.25 mmol) in aqueous sodium hydroxide (2 M, 71 mL) at 0° C. (ice-bath.) The mixture was stirred at 0° C. for an additional 30 minutes, and at room temperature for 2.5 h to complete the reaction. After filtering, the reaction solution was neutralized to pH 7 with 20% hydrochloric acid, diluted with 100 mL of ethanol, and then extracted with ethyl acetate. The extract was washed with brine, dried with MgSO₄ and concentrated in vacuo to afford 12.5 g (95%) of 5b as a yellow solid, LC-MS 210 (MH⁺−H₂O.)

Step 5B:

A suspension of 5b (12.4 g, 0.055 mol) and hydrazine (367 g, 1.15 mol) in 246 mL of water was heated at 95° C. for 3 h. The yellow solid that precipitated was filtered and washed with water. This crude material was triturated with hot ethyl acetate to afford 4.1 g (34%) of 5c as a yellow solid, LC-MS 225 (MH⁺.)

Step 5C:

Nitrogen was bubbled through a suspension of 5c (1.41 g, 6.3 mmol), 4-(trifluoromethyl)phenylboronic acid (1.44 g, 7.6 mmol) and potassium carbonate in 35 mL of DMF and 3.5 mL of water for 10 minutes, and then [1,1′-bis(diphenylphosphino)-ferrocene]dichloropalladium(II) DCM complex (0.46g, 0.63 mmol) was added. After the reaction mixture was heated at 80° C. for 15 hours and filtered through Celite, it was partitioned between 100 mL of water and 150 mL of ethyl acetate. The organic extract was washed with brine, dried with MgSO₄ and concentrated in vacuo to a volume of ca. 10 mL. The resulting yellow solid was isolated by filtration to afford 0.31 g (25%) of 5d. The mother liquid was concentrated and purified by flash column chromatography (elution with 2% methanol and 0.5% aqueous ammonia in DCM) to afford an additional 0.09 g (6%) of 5d, LC-MS 291 (MH⁺.)

Step 5D:

A suspension of 5d (116 mg, 0.40 mmol), compound 1-1 (130 mg, 0.48 mmol), cesium carbonate (261 mg, 0.80 mmol), copper(I) iodide (4 mg, 0.02 mmol) and trans-1,2-cyclohexanediamine (5 mg, 0.04 mmol) in 4 mL of dioxane was heated at 115° C. for 15 hours. After filtering, the solution was partitioned between 10 mL of DCM and 4 mL of water. The DCM extract was washed with brine, dried with MgSO₄ and concentrated in vacuo to obtain the crude compound. This material was recrystallized from ethyl acetate to afford 203 mg (85%) of 5-1, LC-MS 480 (MH⁺.)

Using the appropriate starting materials, the following compounds were prepared according to the above procedures.

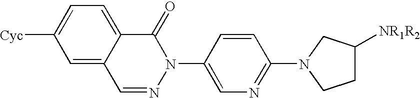

| No. | Cyc | NR₁R₂ | MW | MH⁺ | t_{R} |
|---|---|---|---|---|---|
| 5-1 | 4-CF₃-phenyl | -N(CH₃)₂ | 479.50 | 479 | 4.802 |
| 5-2 | 4-Cl-phenyl | -N(CH₃)₂ | 445.95 | 446 | 1.803 |
| 5-3 | 4-methoxy-2-methyl- | -N(CH₃)₂ | 455.56 | 455 | 4.617 |
| | phenyl | | | | |
| 5-4 | 4-Cl-phenyl | (5)-pyrrolidin-1-yl | 471.99 | 472 | 4.820 |
| 5-5 | 4-Cl-phenyl | (S)-N(H)CH₂CH₃ | 445.95 | 446 | 4.731 |
| 5-6 | 4-CF₃-phenyl | (S)-pyrrolidin-1-yl | 505.54 | 506 | 4.944 |
| 5-7 | 4-CF₃-phenyl | (S)-N(H)CH₂CH₃ | 479.50 | 480 | 4.853 |
| 5-8 | 4-Cl-phenyl | (S)-N(H)CH₃ | 431.92 | 432 | 1.849 |
| 5-9 | 4-Cl-phenyl | (R)-N(H)CH₃ | 431.92 | 432 | 1.865 |
| 5-10 | 4-Cl-phenyl | (S)-N(CH₃)₂ | 445.95 | 446 | 2.035 |
| 5-11 | 4-Cl-phenyl | (R)-N(CH₃)₂ | 445.95 | 446 | 2.017 |
| 5-12 | 4-Cl-phenyl | (S)-N(CH₃)CH₂CH₃ | 459.98 | 460 | 1.968 |
| 5-13 | 4-Cl-phenyl | (R)-N(CH₃)CH₂CH₃ | 459.98 | 460 | 1.968 |
| 5-14 | 4-Cl-phenyl | (R)-pyrrolidin-1-yl | 471.99 | 472 | 1.997 |
| 5-15 | 4-CF₃-phenyl | (R)-N(H)CH₃ | 465.48 | 466 | 1.806 |
| 5-16 | 4-CF₃-phenyl | (S)-N(CH₃)₂ | 479.50 | 480 | 1.951 |
| 5-17 | 4-CF₃-phenyl | (R)-N(CH₃)₂ | 479.50 | 480 | 1.824 |
| 5-18 | 4-CF₃-phenyl | (S)-N(CH₃)CH₂CH₃ | 493.53 | 494 | 1.894 |
| 5-19 | 4-CF₃-phenyl | (R)-N(CH₃)CH₂CH₃ | 493.53 | 494 | 1.67 |
| 5-20 | 4-CF₃-phenyl | (S)-N(CH₂CH₃)₂ | 507.56 | 508 | 1.863 |
| 5-21 | 4-CF₃-phenyl | (R)-pyrrolidin-1-yl | 505.54 | 506 | 1.862 |
| 5-22 | 4-methoxy-2-methyl- | (S)-N(H)CH₃ | 441.53 | 442 | 1.788 |
| | phenyl | | | | |
| 5-23 | 4-methoxy-2-methyl- | (R)-N(H)CH₃ | 441.53 | 442 | 4.189 |
| | phenyl | | | | |
| 5-24 | 4-methoxy-2-methyl- | (S)-N(CH₃)₂ | 455.56 | 456 | 1.922 |
| | phenyl | | | | |
| 5-25 | 4-methoxy-2-methyl- | (R)-N(CH₃)₂ | 455.56 | 456 | 1.751 |
| | phenyl | | | | |
| 5-26 | 4-methoxy-2-methyl- | (S)-N(H)CH₂CH₃ | 455.56 | 456 | 1.718 |
| | phenyl | | | | |
| 5-27 | 4-methoxy-2-methyl- | (S)-N(CH₃)CH₂CH₃ | 469.59 | 470 | 1.884 |
| | phenyl | | | | |
| 5-28 | 4-methoxy-2-methyl- | (R)-N(CH₃)CH₂CH₃ | 469.59 | 470 | 1.623 |
| | phenyl | | | | |
| 5-29 | 4-methoxy-2-methyl- | (S)-N(CH₂CH₃)₂ | 483.61 | 484 | 1.846 |
| | phenyl | | | | |
| 5-30 | 4-methoxy-2-methyl- | (S)-pyrrolidin-1-yl | 481.60 | 482 | 1.897 |
| | phenyl | | | | |
| 5-31 | 4-methoxy-2-methyl- | (R)-pyrrolidin-1-yl | 481.60 | 482 | 1.469 |
| | phenyl | | | | |
| 5-32 | 4-CF₃-phenyl | (S)-N(H)CH₃ | 465.48 | 466 | 1.782 |
| 5-33 | 4-Cl-phenyl | (R)-N(CH₃)CH₂CH₂OCH₃ | 490.00 | 490 | 5.166 |
| 5-34 | 4-Cl-phenyl | (S)-morpholin-4-yl | 487.99 | 488 | 5.147 |
| 5-35 | 4-Cl-phenyl | (S)-N(CH₂CH₃)₂ | 474.00 | 474 | 1.938 |
| 5-36 | 4-Cl-phenyl | (R)-N(H)C(O)CH₃ | 459.94 | 460 | 1.684 |

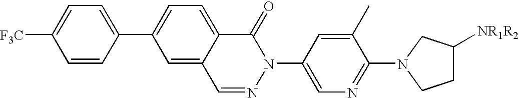

| No. | NR₁R₂ | MW | MH⁺ | t_{R} |
|---|---|---|---|---|
| 5-37 | (R)-N(H)CH₃ | 479.50 | 480 | 4.940 |
| 5-38 | (S)-N(H)CH₃ | 479.50 | 480 | 4.981 |

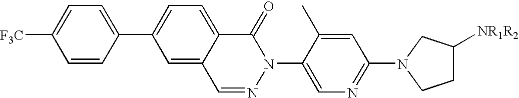

| No. | NR₁R₂ | MW | MH⁺ | t_{R} |
|---|---|---|---|---|
| 5-39 | (R)-N(H)CH₃ | 479.50 | 480 | 4.529 |

### Example 6

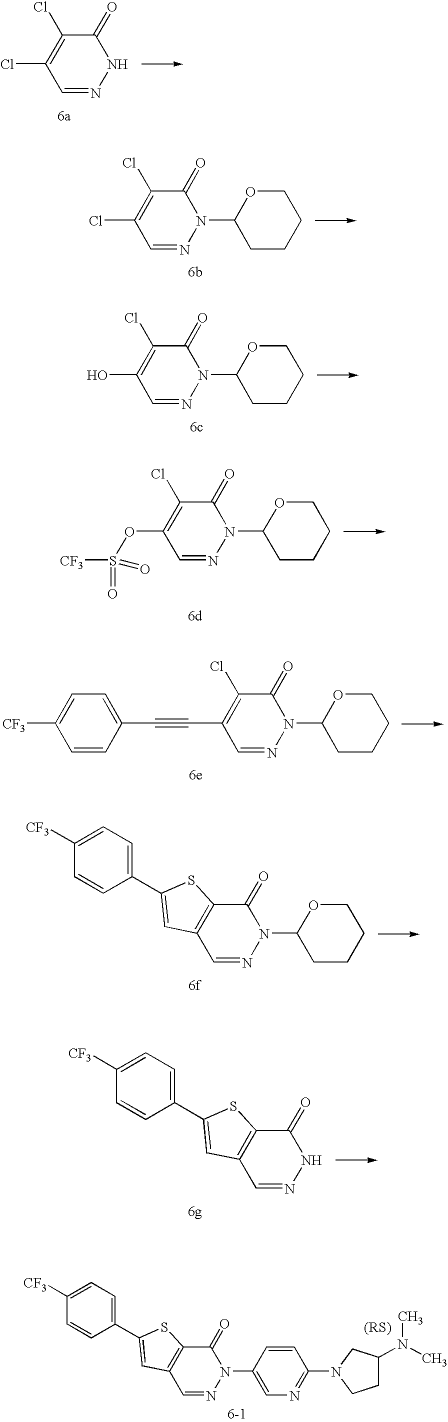 Step 6A:

Compound 6a (10 g, 61 mmol) was suspended in THF (50 mL), and then treated with 3,4-dihydro-2H-pyran (7.0 mL, 77 mmol) and p-toluenesulfonic acid monohydrate (0.94 g, 4.9 mmol.) The mixture was heated to reflux for 2 d, with additional quantities of 3,4-dihydro-2H-pyran being added at 16 h (5.0 mL, 55 mmol) and 24 h (3.0 mL, 33 mmol.) The mixture was concentrated under vacuum, diluted with ethyl acetate (50 mL), and washed twice with aqueous sodium hydroxide (2 N, 20 mL.) The mixture was dried (MgSO₄) and concentrated. The residue was purified by flash chromatography (elution with 15% ethyl acetate in hexanes) to afford 6.0 g (39%) of 6b as a pale yellow oil which solidified on standing.

Step 6B:

Pyridazone 6b (6.0 g, 24 mmol) was dissolved in ethylene glycol (94 mL) and treated with potassium hydroxide (3.95 g, 70 mmol.) The mixture was heated at 130° C. for 3 h, cooled, and poured into water (200 mL.) The mixture was extracted four times with DCM (50 mL), and the combined extracts were dried (MgSO₄) and concentrated to afford 4.85 g (88%) of 6c as a brown oil.

Step 6C:

Compound 6c (2.73 g, 11.8 mmol) and TEA (2.5 mL, 18 mmol) were dissolved in DCM (80 mL) and cooled in an ice bath. Trifluoromethanesulfonic anhydride (2.4 mL, 14 mmol) was added over five minutes, and the mixture was stirred at 0° C. for 30 minutes. The mixture was poured into 0.5 M hydrochloric acid (50 mL) and extracted three times with DCM (50 mL.) The combined extracts were washed with 1% aqueous sodium bicarbonate (50 mL) and aqueous sodium chloride (30 mL), dried (MgSO₄), and concentrated under vacuum to afford 3.84 g (90%) of 6d as a brown oil.

Step 6D:

Sulfonate 6d (1.48 g, 4.08 mmol) was dissolved in THF (22 mL.) Dichlorobis(triphenylphosphine)palladium(II) (84 mg, 0.12 mmol), copper(I) iodide (226 mg, 1.19 mmol) and tetra-n-butylammonium iodide (4.51 g, 12.2 mmol) were added and the mixture was stirred for 10 seconds prior to the addition of TEA (1.55 mL, 11.1 mmol) and 4-(trifluoromethyl)phenylacetylene (0.67 mL, 4.1 mmol.) The mixture was stirred for 3 h, ethyl acetate (44 mL) was added, and the mixture was filtered through a pad of Celite®. The mixture was concentrated under vacuum and the residue was purified by flash chromatography (elution with 20% ethyl acetate in hexanes) to afford 467 mg (82%) of 6e as a white powder, LC-MS 299 (MH⁺.)

Step 6E:

Compound 6e (466 mg, 1.22 mmol) was dissolved in DMF (6 mL), sodium sulfide nonahydrate (643 mg, 2.68 mmol) was added, and the mixture was heated at 70° C. for 1 h. The mixture was poured into water (24 mL) and extracted four times with DCM (15 mL.) The combined extracts were dried (MgSO₄,) concentrated under vacuum, and the residue was purified by flash chromatography (elution with 20% ethyl acetate in hexanes) to afford 422 mg (91%) of 6f as a yellow powder, LC-MS 380 (MH⁺.)

Step 6F:

Compound 6f (422 mg, 1.11 mmol) was dissolved in methanol (15 mL) and treated with 6 M hydrochloric acid (120 mL.) The mixture was heated at 80° C. for 2 h, cooled, and extracted four times with DCM (40 mL.) The combined extracts were dried (MgSO₄) and concentrated to afford 248 mg (75%) of the thienopyridazone 6g as a red powder, LC-MS 297 (MH⁺).

Alternatively 6g may be made according to the following procedure:

A mixture of sodium carbonate (383.9 g, 3.62 mol) and distilled water (4 L) was heated at 80° C. for 40 min. 5-Bromothiophene-2-carboxylic acid (250 g, 1.21 mol) was added and after 5 min, 4-trifluoromethylbenzeneboronic acid (240.8 g, 1.27 mol) was added. Palladium acetate (0.542 g, 0.00241 mol) was added and the reaction mixture was heated at 80° C. for 18 h. Palladium acetate (0.27 g, 0.0012 mol) and 4-trifluoromethylbenzeneboronic acid (11.5 g, 0.061 mol) were added and heating was continued for 24 h. Additional palladium acetate (0.27 g, 0.0012 mol) and 4-trifluoromethylbenzeneboronic acid (11.5 g, 0.061 mol) were added and heating was continued for another 72 h. The reaction mixture was allowed to cool below 50° C. and concentrated HCl solution (500 mL) was added dropwise over 5 h. After overnight stirring, the solid was collected by filtration and washed with water (5×500 mL) until the washing filtrate was colorless. Toluene (8 L) was added to the solid and residual water in the crude product was removed azeotropically at 52° C. under house vacuum. The solid was collected by filtration and dried to afford 5-(4-trifluoromethylphenyl)-thiophene-2-carboxylic acid (279.1 g, 85%) as a grey solid. MS 273 (MH⁺).

5-(4-Trifluoromethylphenyl)-thiophene-2-carboxylic acid (100 g, 0.367 mol) and anhydrous THF (1.8 L) were cooled to −40° C. to −50° C. n-BuLi in hexane (2.5M, 320 mL, 0.81 mol) was added over 2 hours maintaining the temperature below −44° C. The reaction mixture was stirred at −52° C. to −40° C. for 8 h. Anhydrous DMF (266 mL, 3.42 mol) was added dropwise over 2 hours maintaining the temperature below −40° C. After stirring for 12 hours at −40° C., the temperature rose to 10° C. over 18 h. The reaction mixture was once again cooled to −20° C. to −27° C. and a 1 N HCl solution (1.47 L) was added over 3 hours, maintaining the temperature below −10° C. EtOAc (1 L) was added and the mixture was allowed to warm to room temperature. The layers were separated and the aqueous layer was extracted with EtOAc (3×1 L). The combined organic layers were washed with water (3×1.5 L), brine (2 L), dried over sodium sulfate, filtered, and concentrated in vacuo to give a brown solid. The brown solid was slurried in toluene (1.2 L), collected by filtration, washed with toluene (3×250 mL), and dried to give 5-(4-fluoro-phenyl)-3-formyl-thiophene-2-carboxylic acid (93.5 g, 86%) as a light brown solid. MS 301 (MH⁺).

To 5-(4-fluoro-phenyl)-3-formyl-thiophene-2-carboxylic acid (93.5 g, 0.31 mol) and EtOH (935 mL) was added hydrazine monohydrate (18.1 mL, 0.37 mol) dropwise over 10 min. Conc. HCl (156 mL, 1.6 mol) was added dropwise at a rate of 3 mL/min and the resulting reaction mixture was heated at 82° C. for 42 h. The reaction mixture was allowed to cool to 43° C. and sat. NaHCO₃ solution (1.3 L) was added at a rate of 10 mL/min until pH 8. The light green solid that resulted was collected by filtration, washed with water (3×250 mL), and dried to give the desired 2-(4-trifluoromethyl-phenyl)-6H-thieno[2,3-d]pyridazin-7-one 6g (88.9 g, 96%). LC-MS 297 (MH⁺).

Step 6G:

Compounds 6g and 1-1 were coupled using the conditions described in Step 5d (Example 5) to afford after purification compound 6-1, LC-MS 486 (MH⁺.)

Using the appropriate starting materials, the following compounds were prepared according to the above procedures.

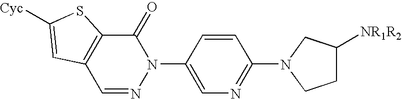

| No. | Cyc | NR₁R₂ | MW | MW⁺ | t_{R} |
|---|---|---|---|---|---|
| 6-1 | 4-CF₃-phenyl | —N(CH₃)₂ | 485.53 | 486 | 5.031 |
| 6-2 | 4-CF₃-phenyl | (R)-N(CH₃)₂ | 485.53 | 486 | 1.965 |
| 6-3 | 4-CF₃-phenyl | (S)-pyrrolidin-1-yl | 511.57 | 512 | 1.967 |
| 6-4 | 4-CF₃-phenyl | (R)-NH(CH₃) | 471.51 | 472 | 1.840 |
| 6-5 | 4-methoxy-phenyl | (S)-pyrrolidin-1-yl | 473.60 | 474 | 2.015 |
| 6-6 | 4-ethyl-phenyl | (S)-pyrrolidin-1-yl | 471.63 | 472 | 1.922 |
| 6-7 | 4-CF₃-phenyl | (R)-pyrrolidin-1-yl | 511.57 | 512 | 5.137 |
| 6-8 | 4-CF₃-phenyl | (R)-N(CH₃)CH₂CH₃ | 499.56 | 500 | 5.134 |
| 6-9 | 4-CF₃-phenyl | (S)-N(CH₃)₂ | 485.53 | 486 | 5.029 |
| 6-10 | 4-CF₃-phenyl | (S)-NH(CH₂CH₃) | 485.53 | 486 | 4.959 |
| 6-11 | 4-CF₃-phenyl | (S)-N(CH₃)CH₂CH₃ | 499.56 | 500 | 5.137 |
| 6-12 | 4-CF₃-phenyl | (S)-N(CH₂CH₃)₂ | 513.58 | 514 | 5.274 |
| 6-13 | 4-methoxy-phenyl | (R)-NH(CH₃) | 433.53 | 434 | 4.1 |
| 6-14 | 4-ethyl-phenyl | (R)-NH(CH₃) | 431.56 | 432 | 4.852 |
| 6-15 | 4-F-phenyl | (R)-NH(CH₃) | 421.50 | 422 | 4.16 |
| 6-16 | 4-F-phenyl | (S)-N(CH₃)CH₂CH₃ | 449.55 | 450 | 4.388 |
| 6-17 | 4-F-phenyl | (S)-NH(CH₂CH₃) | 435.52 | 436 | 4.269 |
| 6-18 | 4-Cl-phenyl | (R)-NH(CH₃) | 437.95 | 438 | 4.712 |
| 6-19 | 4-Cl-phenyl | (S)-N(CH₃)CH₂CH₃ | 466.01 | 466 | 4.911 |
| 6-20 | 4-Cl-phenyl | (S)-NH(CH₂CH₃) | 451.98 | 452 | 4.747 |
| 6-21 | 4-Cl-phenyl | (S)-pyrrolidin-1-yl | 478.02 | 478 | 4.885 |
| 6-22 | 4-Cl-phenyl | (S)-N(CH₃)₂ | 451.98 | 452 | 4.771 |
| 6-23 | 4-F-phenyl | (R)-N(CH₃)CH₂CH₂OCH₃ | 479.58 | 480 | 4.392 |
| 6-24 | 4-F-phenyl | (R)-(4-acetyl-piperazin-1-yl) | 518.61 | 519 | 4.41 |
| 6-25 | 4-F-phenyl | 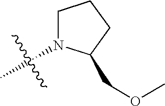 | 505.62 | 506 | |
| 6-26 | 4-F-phenyl | (R)-morpholin-4-yl | 477.56 | 478 | 4.399 |
| 6-27 | 4-Cl-phenyl | 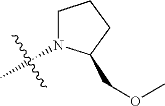 | 522.07 | 522 | 5.063 |
| 6-28 | 4-Cl-phenyl | (R)-morpholin-4-yl | 494.02 | 494 | 4.843 |
| 6-29 | 4-F-phenyl | (S)-NH(CH₃) | 421.50 | 422 | 4.316 |
| 6-30 | 4-Cl-phenyl | (S)-N(CH₃)₂ | 437.95 | 438 | 4.629 |
| 6-31 | 4-CF₃-phenyl | (S)-N(CH₃)₂ | 471.50 | 472 | 4.988 |
| 6-32 | 4-F-phenyl | (R)-N(CH₃)₂ | 435.52 | 436 | 4.325 |
| 6-33 | 4-F-phenyl | (S)-pyrrolidin-1-yl | 461.56 | 462 | 4.461 |
| 6-34 | 4-F-phenyl | (S)-N(CH₃)₂ | 435.52 | 436 | 4.371 |
| 6-35 | 4-methoxy-2-methyl- | (R)-NH(CH₃) | 447.56 | 448 | 4.220 |
| | phenyl | | | | |
| 6-36 | 4-CF₃-phenyl | (R)-morpholin-4-yl | 527.57 | 528 | 4.936 |

### Example 7

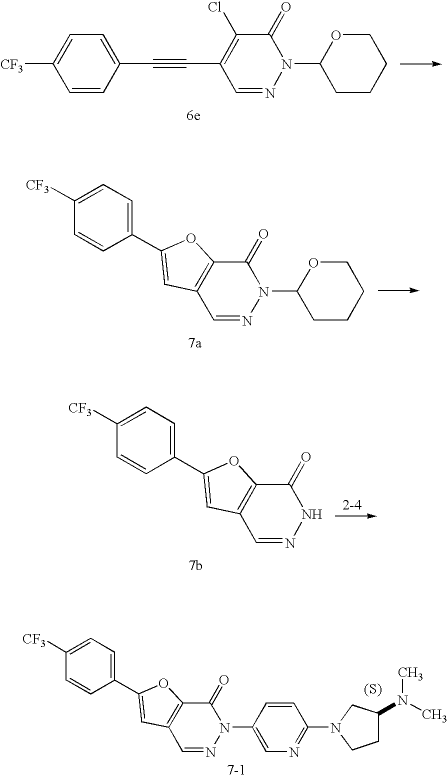 Step 7A:

Alkyne 6e (363 mg, 0.948 mmol) was dissolved in dioxane (9 mL) and potassium hydroxide (710 mg, 12.7 mmol) in water (4.5 mL) was added. The mixture was heated to reflux for 3 h, cooled to room temperature, and poured into water (60 mL). The mixture was extracted three times with ethyl acetate and the combined extracts were dried (MgSO₄) and concentrated. The residue was purified by flash chromatography (elution with 25% ethyl acetate in hexanes) to afford 135 mg (37%) of 7a as a yellow solid. LC-MS 365 (MH⁺).

Step 7B:

Compound 7b was prepared from 7a using the procedure of Step 6F.

Step 7C:

Compound 7b and compound 2-4 were coupled using the conditions described in Step 5d to afford compound 7-1, LC-MS 470 (MH⁺).

### Example 8

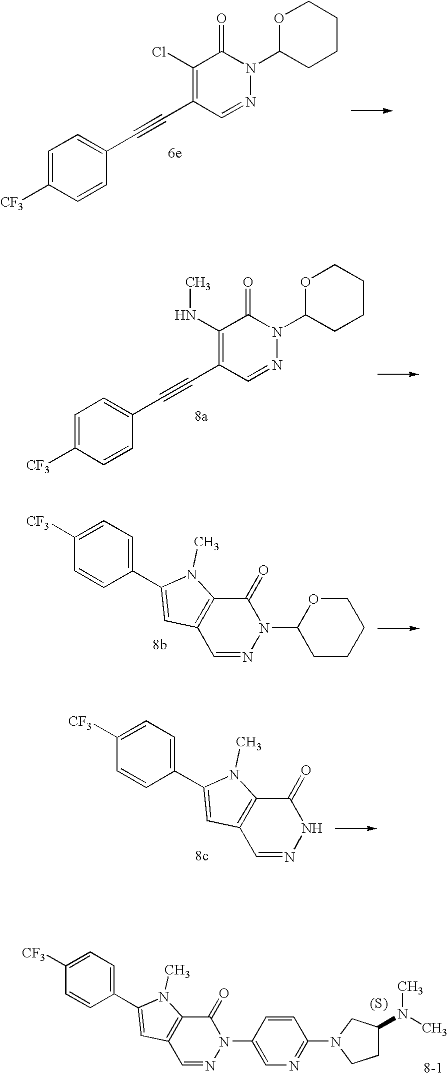 Step 8A:

Alkyne 6e (383 mg, 1.00 mmol) was suspended in ethanol (20 mL) and treated with 40% aqueous methylamine (6.1 mL). The mixture was heated in a sealed tube at 80° C. for 2 h and cooled to room temperature. The resulting precipitate was isolated by filtration and washed with cold ethanol to afford 162 mg (47%) of 8a as white needles, LC-MS 378 (MH⁺).

Step 8B:

Pyridazinone 8a (122 mg, 0.32 mmol) was suspended in dimethylacetamide (3 mL) and ethylene glycol (0.3 mL). Sodium hexamethyldisilazane (90 mg, 0.49 mmol) was added and the mixture was heated in a sealed tube at 130° C. for 18 h. The mixture was cooled to room temperature, diluted with ethyl acetate (15 mL), and washed twice with water and twice with aqueous sodium chloride. It was then dried (MgSO₄), concentrated, and the residue was purified by flash chromatography (elution with 25% ethyl acetate in hexanes) to afford 52 mg (43%) of 8b as a colorless oil, LC-MS 378 (MH⁺).

Step 8C:

Compound 8c was prepared from 8b using the procedure of Step 6F.

Step 8D:

Compound 8c and compound 2-4 were coupled using the conditions described in Step 5d to afford compound 8-1, LC-MS 483 (MH⁺).

### Example 9

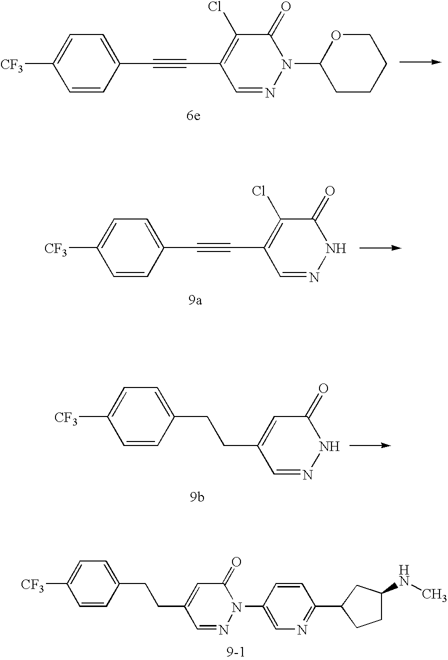 Step 9A:

Compound 6e (200 mg) was deprotected using the methanol and hydrochloric acid conditions described in Step 6F (EXAMPLE 6) to afford compound 9a. This material was dissolved in dioxane (4 mL), palladium on carbon (10%, 10 mg) was added, and the mixture was stirred under a hydrogen atmosphere for 18 h. The mixture was filtered (Celite) and concentrated. The residue was taken up in dichloromethane (2 mL) and stirred with polystyrene-HCO₃ resin (50 mg) for 3 h. The mixture was filtered and concentrated to afford 17 mg (35%) of 9b as a white powder. LC-MS 269 (MH⁺).

Step 9B:

Compounds 9b and 2-5 were coupled using the conditions described in Step 5D (EXAMPLE 5) to afford after purification compound 9-1. LC-MS 444 (MH⁺).

### Example 10

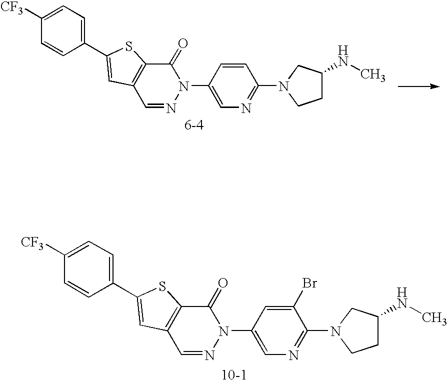 Step 10A:

Example 6-4 (920 mg, 1.95 mmol) was dissolved in DMF (34 mL) and treated with N-bromosuccinimide (1.31 g, 7.33 mmol). After 20 h, the mixture was poured into 1 N aqueous sodium hydroxide and then extracted three times with DCM. The combined extracts were dried (MgSO₄), concentrated, and the residue was purified by flash chromatography (elution with 1% methanol and 0.5% aqueous ammonia in DCM) to afford 180 mg (17%) of 10-1 as an orange solid. LC-MS 550 (MH⁺).

### Example 11

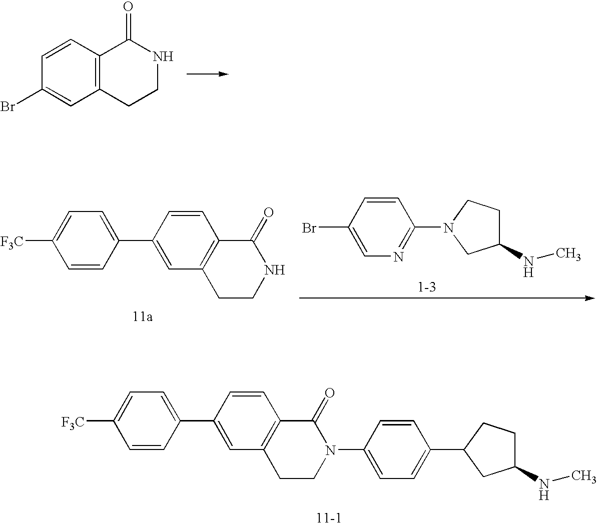 Step 11A:

A mixture of 6-bromo-3,4-dihydro-2H-isoquinolin-1-one (250 mg, 1.11 mmol), 4-trifluoromethyl-phenyl-boronic acid (251 mg, 1.33 mmol), 2 M aqueous sodium carbonate (1.10 mL), toluene (6 mL), ethanol (2 mL) and water (1 mL) was purged with nitrogen for 5 minutes. Palladium-tetrakis (triphenylphosphine) (64 mg, 0.055 mmol) was added and the mixture was heated with stirring at 80° C. in a sealed pressure vessel for 19 h. Water (1 mL) was added and the reaction mixture was extracted with ethyl acetate (3×5 mL). The combined organic fractions were rinsed with water (10 mL) and concentrated in vacuo to give 475 mg of a light orange solid. Chromatography (silica gel 4: 96:0.5, methanol: CH₂Cl₂:NH₄OH) afforded 189 mg (59%) of 11a as an off-white solid. LC-MS 292.0 (MH⁺).

Step 11B:

Compounds 11a and 1-3 were coupled using the conditions described in Step 5D (EXAMPLE 5) to afford after purification compound 11-1. LC-MS 467.1 (MH⁺).

Using the appropriate starting materials, the following compounds were prepared according to the above procedures.

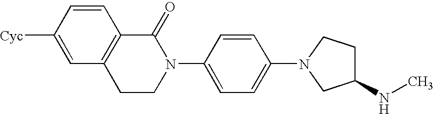

| No. | Cyc | MW | MH⁺ | t_{R} |
|---|---|---|---|---|
| 11-1 | 4-CF₃-phenyl | 466.5 | 467.1 | 4.690 |
| 11-2 | 4-Cl-phenyl | 432.9 | 433.0 | 5.140 |

### Example 12

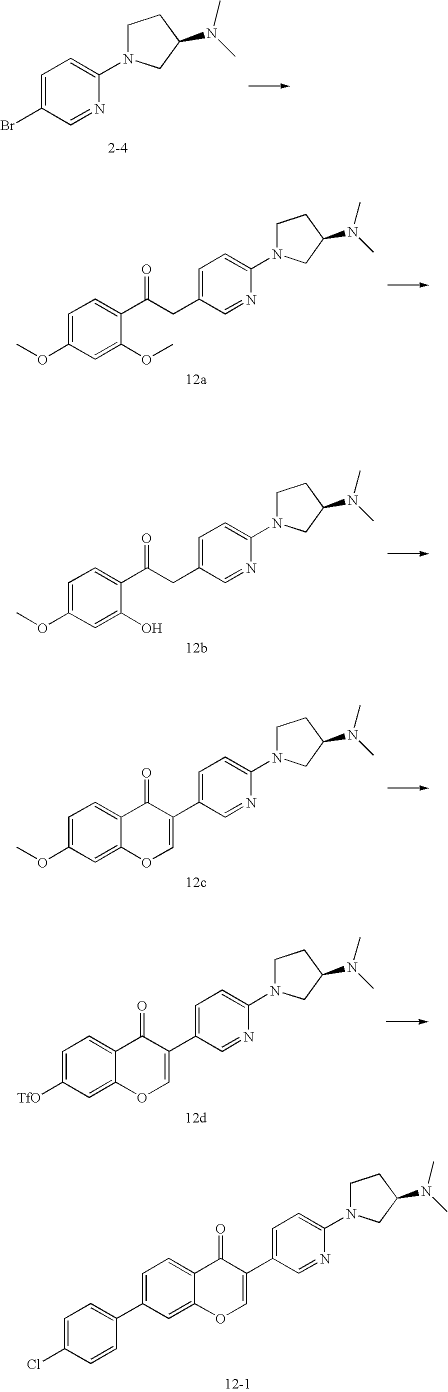 Step 12A:

To a solution of 2,4-dimethoxyacetophenone (59.0 g, 328 mmol) and bromide 2-4 (43.2 g, 161 mmol) in dry tetrahydrofuran (1 L) under nitrogen was added 2-(dicyclohexylphosphino)-2′-methylbiphenyl (0.590 g, 1.60 mmol). The mixture was degassed for 15 min. Sodium tert-butoxide (39.2 g, 408 mmol), followed by palladium acetate (0.180 g, 0.800 mmol) were then added. The mixture was refluxed while maintaining vigourous stirring for 15 h. The mixture was then concentrated and treated with 2 M aqueous HCl (500 mL) and dichloromethane (1 L). After separating, the dichloromethane layer was extracted with 2 M HCl (2×500 mL). The combined aqueous layers were taken to pH 10 with 50% aqueous sodium hydroxide, then were extracted with dichloromethane (3×500 mL). The combined organics were washed with brine (500 mL), dried over magnesium sulfate, and concentrated. Purification by column chromatography (1:99 methanol/dichloromethane to 0.5:5:94.5 ammonium hydroxide/methanol/dichloromethane) gave 12a as a viscous yellow oil (41.4 g, 70%). ¹H-NMR (300 MHz, CDCl₃) δ7.99 (d, J=2.4 Hz, 1H), 7.78 (d, J=9.0 Hz, 1H), 7.34 (dd, J=8.7, 2.4 Hz, 1H), 6.50 (dd, J =8.7, 2.4 Hz, 1H), 6.44 (d, J =2.4 Hz, 1H), 6.31 (d, J =8.7 Hz, 1H), 4.11 (s, 2H), 3.91 (s, 3H), 3.84 (s, 3H), 3.70-3.84 (m, 1H), 3.54-3.64 (m, 1H), 3.32-3.43 (m, 1H), 3.18-3.25 (m, 1H), 2.72-2.84 (m, 1H), 2.31 (s, 6H), 2.16-2.28 (m, 1H), 1.82-1.98 (m, 1H); MS m/z: 370.0 ([M+H]⁺).

Step 12B:

Sodium iodide (50.4 g, 336 mmol) and 12a (41.4 g, 112 mmol) were dissolved in dry acetonitrile (500 mL) under nitrogen and cooled to 0° C., with vigourous stirring. Aluminum trichloride (44.7 g, 336 mmol) was added in four portions, allowing the internal temperature to cool to <5° C. prior to each addition. Following the last addition, the mixture was stirred for 30 min, then warmed to room temperature and stirred for an additional 4 h. The mixture was concentrated and treated with 1.2 M aqueous HCl (1 L). The aqueous layer was extracted with diethyl ether (3×500 mL), made basic (pH 10) with 50% aqueous sodium hydroxide, and extracted with dichloromethane (2×500 mL). The organic layer was washed with brine (500 mL), dried over magnesium sulfate, and evaporated to yield 12b as a yellow solid (32.0 g, 80%). An analytical sample was obtained by triturating the solid with diethyl ether (3×). ¹H-NMR (300 MHz, CDCl₃) δ12.68 (s, 1H), 8.04 (d, J=1.8 Hz, 1H), 7.75 (d, J=9.0 Hz, 1H), 7.37 (dd, J=8.4, 2.4 Hz, 1H), 6.40-6.46 (m, 2H), 6.34 (d, J=6.9 Hz, 1H), 4.06 (s, 2H), 3.82 (s, 3H), 3.71-3.79 (m, 1H), 3.56-3.65 (m, 1H), 3.32-3.47 (m, 1H), 3.19-3.27 (m, 1H), 2.75-2.84 (m, 1H), 2.31 (s, 6H), 2.17-2.28 (m, 1H), 1.83-1.99 (m, 1H); MS m/z: 356.0 ([M+H]⁺).

Step 12C:

To a stirred solution of phenol 12b (30.4 g, 85.6 mmoL) in dichloromethane (300 mL) was added DMF-DMA (100 mL, 856 mmoL). Stirring was continued for 2 h. The mixture was concentrated and purified by column chromatography (1:99 methanol/dichloromethane to 0.5:5:94.5 ammonium hydroxide/methanol/dichloromethane) to give the chromone 12c as a tan solid (29.6 g, 95%). ¹H-NMR (300 MHz, CDCl₃) δ8.17-8.22 (m, 2H), 7.90 (s, 1H), 7.80 (dd, J=8.7, 2.4 Hz, 1H), 6.97 (dd, J=8.7, 2.4 Hz, 1H), 6.84 (d, J=2.4 Hz, 1H), 6.43 (d, J=8.7 Hz, 1H), 3.91 (s, 3H), 3.78-3.85 (m, 1H), 3.64-3.72 (m, 1H), 3.39-3.48 (m, 1H), 3.25-3.31 (m, 1H), 2.78-2.88 (m, 1H), 2.31 (s, 6H), 2.20-2.31 (m, 1H), 1.87-2.01 (m, 1H); MS m/z: 366.0 ([M+H]⁺).

Step 12D:

A mixture of chromone 12c (33.1 g, 90.0 mmol) and sodium iodide (20.0 g, 135 mmol) in 48% HBr (800 mL) was stirred and heated at 110° C. in a sealed pressure vessel for 24 h. Additional sodium iodide (20 g, 135 mmol) was added, and heating was continued for 24h. The mixture was concentrated, then methanol (700 mL) and triethylamine (150 mL) were added. The mixture was concentrated to give a purple solid. The solid was washed with dichloromethane (700 mL), then triturated with hot isopropanol (500 mL) to give a light pink solid (44.6 g) which still contained inorganic salts.

This solid was suspended in dry dichloromethane (1 L) under nitrogen, and triethylamine (52 mL, 375 mmol) was added. The mixture was cooled to 0° C., then trifluoromethanesulfonic anhydride was added in 5 mL portions (22 mL total, 126 mmol) until the reaction was complete. The mixture was quenched at 0° C. with saturated aqueous sodium bicarbonate (500 mL) and the layers separated. The aqueous layer was extracted with dichloromethane (500 mL), and the combined organics were washed with brine (500 mL), dried over magnesium sulfate, and concentrated to give a yellow solid. Trituration with diethyl ether provided triflate 12d as a pale yellow solid (37.1 g, 85%, 2 steps). ¹H-NMR (300 MHz, CDCl₃) δ8.37 (d, J=9.0 Hz, 1H), 8.24 (d, J=1.8 Hz, 1H), 8.04 (s, 1H), 7.77 (dd, J=8.1, 2.4 Hz, 1H), 7.46 (d, J=2.4 Hz, 1H), 7.33 (dd, J=8.7, 2.4 Hz, 1H), 6.48 (d, J=8.7 Hz, 1H), 3.90-3.98 (m, 1H), 3.61-3.76 (m, 2H), 3.42-3.54 (m, 1H), 2.78 (s, 6H), 2.70-2.81 (m, 1H), 2.40-2.58 (m, 1H), 2.26-2.35 (m, 1H); MS m/z: 483.9 ([M+H]⁺).

Step 12E:

A mixture of triflate 12d (3.22 g, 6.66 mmol), 4-chlorophenylboronic acid (1.03 g, 6.66 mmol) and potassium carbonate (2.76 g, 20.0 mmol) in dimethylacetamide (50 mL) was degassed for 20 min in a sealed vessel. Dichloro (1,1-bis(diphenylphosphino) ferrocene) palladium(II), (Pd(dppf)Cl₂, 0.109 g, 0.133 mmol) was added and the mixture was stirred and heated at 80° C. for 15 h. The mixture was concentrated, diluted with dichloromethane (300 mL), washed with saturated aqueous sodium bicarbonate (300 mL), water (300 mL) and brine (300 mL). The organic layer was dried over magnesium sulfate, concentrated and purified by via column chromatography (0.5:5:94.5 ammonium hydroxide/methanol/dichloromethane) to give a tan solid (1.82 g, 61%). Residual palladium was scavenged by dissolving the solid (1.82 g) in 9:1 dichloromethane/methanol (120 mL), adding macroporous polystyrene-2,4,6-trimercaptotriazine (MP-TMT, 0.70 g), and stirring for 40 h, then filtering and concentrating. An analytical sample of 12-1 was obtained by trituration with hot tert-butylmethyl ether. ¹H-NMR (300 MHz, DMSO-D6) δ8.57 (s, 1H), 8.31 (d, J=2.4 Hz, 1H), 8.20 (d, J=8.1 Hz, 1H), 8.02 (s, 1H), 7.92 (s, 1H), 7.89 (s, 1H), 7.85 (dd, J=8.4, 1.8 Hz, 1H), 7.78 (dd, J=8.7, 2.4 Hz, 1H), 7.62 (s, 1H), 7.59 (s, 1H), 6.54 (d, J=8.7 Hz, 1H), 3.67-3.76 (m, 1H), 3.58-3.67 (m, 1H), 3.14-3.42 (m, 2H), 2.82-2.94 (m, 1H), 2.26 (s, 6H); 2.12-2.36 (m, 1H), 1.80-1.89 (m, 1H); APCI MS m/z: 446.1 ([M+H]⁺). The HCl salt was obtained by dissolving 12-1 in a minimum amount of 20:1 dichloromethane/methanol, and adding an excess of 2M HCl in diethyl ether (3 eq.) to give a cream solid which was collected by filtration and rinsed with diethyl ether.

### Example 13

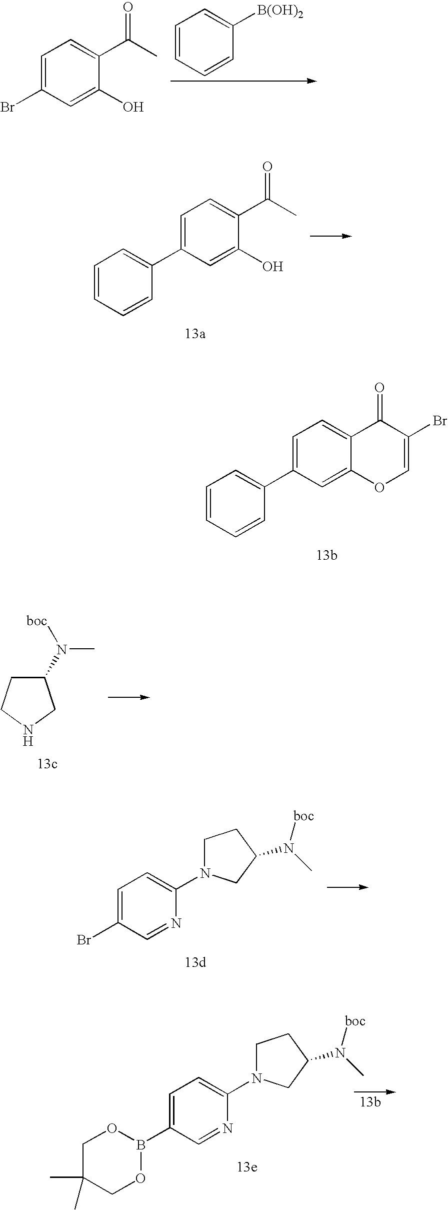 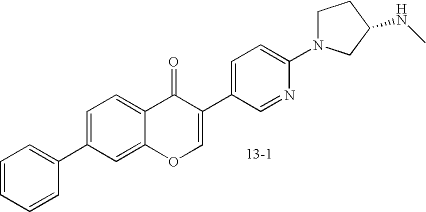 Step 13A:

A mixture of 4-bromo-2-hydroxyacetophenone (6.10 g, 28.5 mmol), phenylboronic acid (3.83 g, 31.9 mmol), sodium carbonate (12.3 g, 116 mol) and palladium(II)acetate (65 mg, 0.29 mmol) in dioxane (50 mL) and water (16 mL) was degassed for 10 min, then vigorously stirred and heated at 90° C. under nitrogen overnight. Aqueous 2M HCl (75 mL) and ethyl acetate (50 mL) were then added. The aqueous layer was further extracted with ethyl acetate (50 mL), and the combined organics were washed with water (50 mL) and brine (50 mL), dried over magnesium sulfate, and evaporated to give a red-brown solid. The solid was purified by column chromatography (20% ethyl acetate/hexane) to give the phenol 13a as a pale yellow solid (5.20 g, 86%). ¹H-NMR (300 MHz, CDCl₃) δ12.36 (s, 1H), 7.79 (d, J=8.1 Hz, 1H), 7.60-7.64 (m, 2H), 7.41-7.50 (m, 3H), 7.22 (d, J=1.8 Hz, 1H), 7.14 (dd, J=8.4, 1.8 Hz, 1H), 2.66 (s, 3H); MS m/z: 213.0 ([M+H]⁺).

Step 13B:

A solution of phenol 13a (3.80 g, 18.0 mmol) in DMF-DMA (35 mL, 260 mmol) was heated at 95° C. for 12 h. The solution was concentrated, and the resulting enamine was triturated in hot MTBE, cooled and isolated as a yellow powder (4.44 g, 92%) which was used immediately in the subsequent reaction. To the enamine (4.44 g, 16.6 mmol) in chloroform (100 mL) was added dropwise bromine (2.93 g, 18.3 mmol), and the reaction was stirred for 1h. The solution was concentrated, diluted with dichloromethane (200 mL), washed with water (100 mL), dried over magnesium sulfate, and evaporated. The residue was triturated in hot MTBE, cooled and filtered to give the bromide 13b as an off-white powder (4.60 g, 92%). ¹H-NMR (300 MHz, CDCl₃) 6 8.32 (d, J=8.1 Hz, 1H), 8.26 (s, 1H), 7.64-7.73 (m, 4H), 7.43-7.54 (m, 3H); MS m/z: 300.8 ([M+H]⁺).

Step 13C:

To a stirred solution of (S)-(+)-3-(methylamino)-1-benzylpyrrolidine (1.54 g, 8.1 mmol) and triethylamine (2.23 mL, 16.0 mmol) in dichloromethane (30 mL) at room temperature and under an inert atmosphere, was added a solution of di-tert-butyl dicarbonate (1.86 g, 8.5 mmol) in dichloromethane (20 mL), dropwise. The solution was stirred for 2 hours then concentrated in vacuo, to afford 2.35 g of a yellow oil. This yellow oil (7.10 g, 0.0245 mol) was combined with 10% palladium on carbon (50% water) (7.84 g, 0.00370 mol) and ammonium formate (9.20 g, 0.147 mol) in ethanol (300 mL) and was heated to reflux for 110 minutes. The mixture was cooled and filtered through celite and washed with additional ethanol. The filtrate was dried with magnesium sulfate and concentrated to afford 3.70 g (76%) of 13c as a clear gum. LC-MS 200 (MH+).

Step 13D:

A mixture of (S)-3-(BOC-methylamino)pyrrolidine 13c (6.56 g, 32.8 mmol), 2,5-dibromopyridine (5.20 g, 21.9 mmol) and p-toluenesulfonic acid (0.84 g, 4.4 mmol) in DMA (10 mL) was heated at 125° C. overnight in a pressure vessel. The mixture was concentrated and partitioned between dichloromethane (100 mL) and 1M aqueous HCl (100 mL). The aqueous layer was taken to pH 10 with 3M aqueous NaOH, then was extracted with dichloromethane (4×100 mL), dried over magnesium sulfate, and concentrated. The residue was purified by column chromatography (2% methanol/dichloromethane) to provide 13d as a white solid (1.50 g, 19%). ¹H-NMR (300 MHz, CDCl₃) δ8.17 (d, J=2.4 Hz, 1H), 7.50 (dd, J=9.3, 2.7 Hz, 1H), 6.27 (d, J=9.3 Hz, 1H), 3.56-3.66 (m, 2H), 3.27-3.43 (m, 2H), 2.81 (s, 3H), 2.04-2.25 (m, 2H), 1.67-1.74 (m, 1H), 1.48 (s, 9H); MS m/z: 356.0 ([M+H]⁺).

Step 13E:

To a stirring solution of the bromide 13d (1.00 g, 2.80 mmol) in THF (10 mL) at −78° C. under nitrogen was added dropwise n-butyllithium (2.5 M in hexanes, 1.4 mL, 3.4 mmol). The mixture was stirred for an additional 20 min, then triisopropyl boronate (1.3 mL, 5.6 mmol) was added in one portion. The mixture was allowed to warm slowly to −50° C., then was recooled to −78° C. Neopentylglycol (0.290 g, 2.80 mmol) was added in one portion, and the mixture was allowed to slowly reach room temperature, and stirred overnight. The mixture was quenched with water (20 mL), extracted with dichloromethane (2×30 mL), dried over magnesium sulfate, and concentrated to give boronate 13e as a yellow solid (0.71 g, 65%) which was used directly in the next reaction without purification. ¹H-NMR (300 MHz, CDCl₃) δ8.53 (d, J=1.8 Hz, 1H), 7.80 (dd, J=8.7, 2.1 Hz, 1H), 6.32 (d, J=8.7 Hz, 1H), 3.73 (s, 4H), 3.32-3.72 (m, 4H), 2.81 (s, 3H), 2.03-2.24 (m, 2H), 1.52-1.69 (m, 1H), 1.48 (s, 9H); 1.01 (s, 6H); MS m/z: 390.1 ([M+H]⁺).

Step 13F:

To a solution of the boronate 13e (0.30 g, 0.77 mmol) in DMF (5 mL) was added bromide 13b (0.155 g, 0.510 mmol), potassium phosphate (0.49 g, 2.3 mmol), and palladium(II) acetate (9 mg, 0.04 mmol). The mixture was heated at 50° C. overnight, then concentrated, filtered, and purified by preparative HPLC-MS to give 13-1 as a pale yellow solid (21 mg, 10%). ¹H-NMR (300 MHz, CD₃OD) δ8.56 (s, 1H), 8.44 (d, J=8.4, 1.8 Hz, 1H), 8.25-8.32 (m, 2H), 7.91 (d, J=1.8 Hz, 1H), 7.84 (dd, J=8.4,1.8 Hz, 1H), 7.76-7.80 (m, 2H), 7.44-7.57 (m, 3H), 7.15-7.19 (m, 1H); 4.02-4.14 (m, 1H), 3.74-3.92 (m, 2H), 3.63-3.65 (m, 2H), 2.85 (s, 3H), 2.59-2.73 (m, 1H), 2.37-2.48 (m, 1H); MS m/z: 398.0 ([M+H]⁺).

### Example 14

### 2-[6-((R)-3-(methylamino)pyrrolidin-1-yl)pyridin-3-yl]-6-phenoxy-2H-phthalazin-1-one

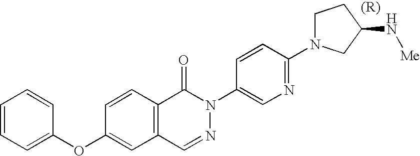 Step 14A:

A mixture of 5c (100 mg, 0.444 mmol), phenol (60 mg, 0.638 mmol), cesium carbonate (289 mg, 0.888 mmol), 2,2,6,6-tetramethyl-3,5-heptanedione (60 mg, 0.326 mmol) and copper (I) chloride (5 mg, 0.044 mmol, 10 mol %) in N-methyl-2-pyrrolidinone (2 mL) was degassed with N₂ over 30 mins. The reaction mixture was heated in a sealed tube at 110° C. for 12 h. After cooling to rt, the reaction mixture was filtered and the filtrate was extracted into a mixture of CHCl₃/IPA (4:1) and washed with sat aq. NaHCO₃ solution. The organic layer was dried (MgSO₄) and the solvent removed to give an oil, which was dissolved in 1,4-dioxane (2 mL). Compound 1-3 (114 mg, 0.444 mmol), cesium carbonate (287 mg, 0.888 mmol), trans-1,2-diaminocyclohexane (50 mg, 0.444 mmol) and copper (I) iodide (8.5 mg, 0.044 mmol, 10 mol %) were added and the reaction mixture was degassed with N₂ over 30 mins and then heated in a sealed tube at 110° C. for 12 h. After cooling to rt, the reaction was quenched with concentrated aq. ammonium hydroxide solution and organics extracted into a mixture of DCM/IPA (4:1). The organic layer was dried (MgSO₄) and the solvent was removed to yield an oil which was purified by preparative LC-MS to give the bis-trifluoroacetate salt of 14-1 (6 mg, 2%) as a colorless solid. LC-MS: 414.0 (MH⁺).

Using the appropriate starting materials, the following compounds were prepared according to the above procedures.

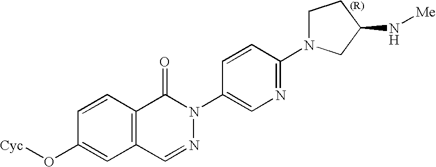

| No. | Cyc | MW | MH⁺ | t_{R} |
|---|---|---|---|---|
| 14-1 | phenyl | 413.48 | 414.0 | 4.851 |
| 14-2 | 4-Cl-phenyl | 447.92 | 448.0 | 4.977 |
| 14-3 | 4-F-phenyl | 431.47 | 432.0 | 4.488 |
| 14-4 | 4-methyl-phenyl | 427.51 | 428.0 | 4.985 |
| 14-5 | 4-methoxy-phenyl | 443.50 | 444.0 | 4.411 |

### Example 15

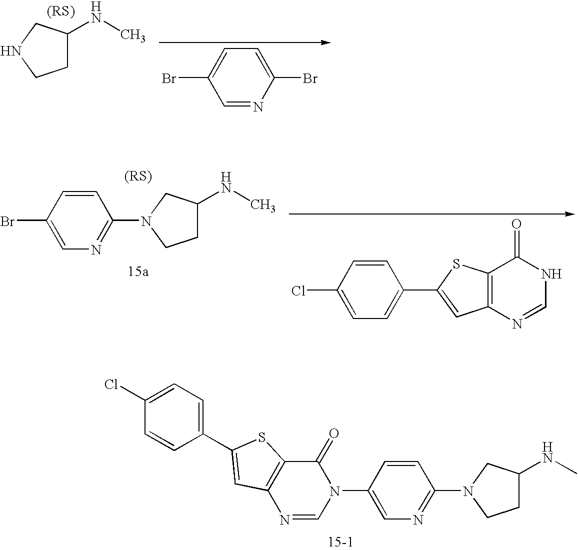 Step 15A:

A mixture of 2,5-dibromopyridine (9.5 g, 40 mmol), 3-methylamino-pyrrolidine (6.0 g, 60 mmol) and TEA (5.6 mL, 40 mmol) in DMA (20 mL) was heated in a sealed tube at 120° C. overnight. The solvent was removed and the residue was diluted with DCM (500 mL). The solution was washed with saturated sodium bicarbonate, brine, dried with MgSO₄, and then concentrated in vacuo. The resulting residue was dissolved in isopropyl alcohol (200 mL), cooled with ice-bath, and 2N HCl in ether (80 mL) was slowly added. The mixture was stored at 4° C. overnight, filtered, and washed with IPA:ether (2:1) to give solid 15.2 g of compound 15a as the HCl salt. The solid was then dissolved in H₂O (50 mL). DCM (400 mL) was added, and the mixture was basified by addition of NaHCO₃ (30g ) in H₂O (200 mL). The DCM layer was separated and the aqueous layer was extracted with IPA:DCM (1:3) 100 mL×3. The combined organic layer was dried over MgSO₄, and then concentrated in vacuo to yield oil. The oil was added DCM (20 mL) and hexanes (80 mL), kept at 4° C. over night to yield some brown solid as impurity. The mother liquid was then concentrated in vacuo to obtain compound 15a as solid (7.8 g). MS: 257 (MH⁺)

[1-(5-Bromo-pyridin-2-yl)-pyrrolidin-3-yl]-(R)-carbamic acid tert-butyl ester 15a.1 was made following the same procedure.

Step 15B:

6-(4-Chloro-phenyl)-3H-thieno[3,2-d]pyrimidin-4-one and 15a were coupled using the conditions described in Step 5D (EXAMPLE 5) to afford 15-1. MS 437.9 (MH⁺)

Using the appropriate starting materials, the following compounds were prepared according to the above procedures.

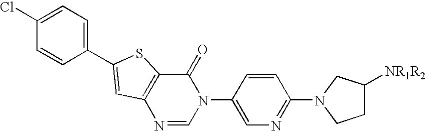

| No. | NR₁R₂ | MW | MH⁺ | t_{R} |
|---|---|---|---|---|
| 15-1 | —NHMe | 437.95 | 438 | 5.620 |
| 15-2 | (R)-NHMe | 437.95 | 438 | 4.879 |

### Example 16

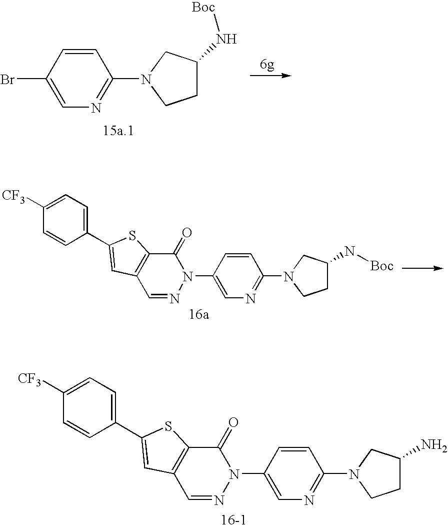 Step 16A:

Compounds 15a.1 and 6g were coupled using the conditions described in Step 5D to give 16a which was deprotected using trifluoroacetic acid/methylene chloride to give 16-1 after purification. MS 457.9 (MH⁺), t_{R}=4.603 min

### Example 17

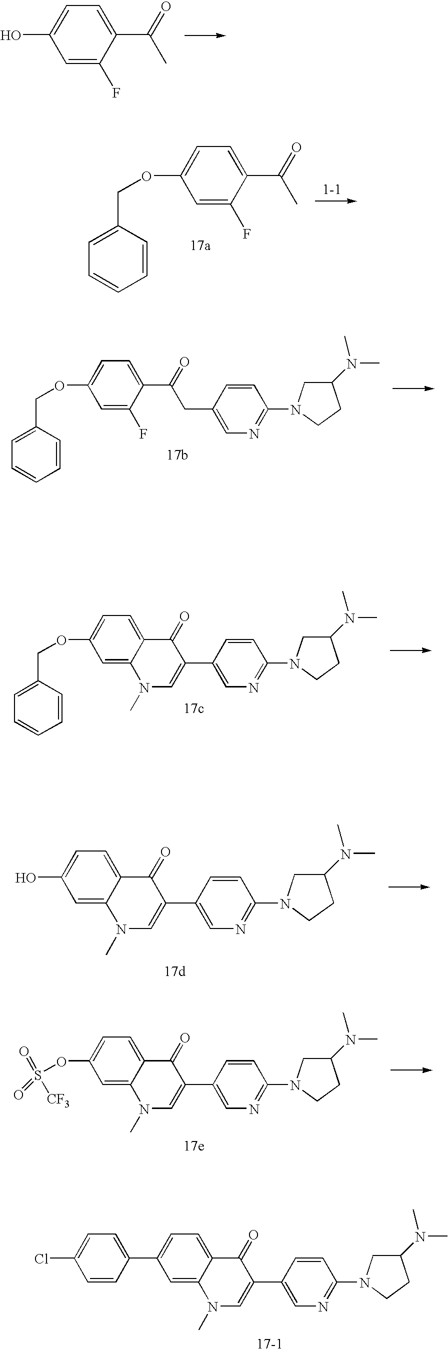 Step 17A:

To a solution of 2′-fluoro-2′-hydroxyacetophenone (9.57 g, 0.062 mol) in 30 mL DMF was added potassium carbonate (17.1 g, 0.124 mol). Benzyl bromide (11.1 mL, 0.093 mol) was added dropwise and the mixture was stirred at room temperature for 4 hours. The DMF was evaporated and the residue was dissolved in 30 mL EtOAc and washed with 1N HCl and brine. The organic layer was dried over MgSO₄ and concentrated. Recrystallization from hexane (30 mL) gave 11.7 g (77%) of 17a.

Step 17B:

Nitrogen was bubbled through a stirred suspension of sodium t-butoxide (5.48 g, 57 mmol), palladium(II) acetate (0.26 g, 1.14 mmol) and 2-(dicyclohexyl phosphino)-2′-methylbiphenyl (0.83 g, 2.28 mmol) in 115 mL of THF for 5 minutes. Compounds 1-1 (6.15 g, 22.8 mmol) and 17a (11.1 g, 45.5 mmol) were added, and the mixture was heated at 80° C. for 2 hours. The mixture was diluted with 100 mL of saturated sodium bicarbonate and then extracted with DCM. The extract was concentrated and the oil was partitioned between diethyl ether and 1N HCl. The ether layer was extracted once with 1N HCl. The pH of the combined aqueous layers was adjusted to 10. The aqueous layer was extracted with DCM, dried over MgSO₄, and concentrated. The crude product was dissolved in diethyl ether and precipitated with the addition of hexane. The precipitate was washed with 4:1 hexane/ether and dried to give 10.3 g (100%) of 17b. MS 434 (MH⁺)

Step 17C:

17b (2.17 g, 10 mmol) was dissolved in 1.32 mL of dimethylformamide dimethylacetal and the reaction was heated to 100° C. After 30 minutes, the excess dimethylformamide dimethylacetal was removed under reduced pressure. The residue was dissolved in 10 mL of ethanol and 5 mL of methylamine (40% in water) and stirred for 30 minutes. The solvent was removed under reduced pressure and the residue was azeotroped twice with ethanol. The residue was dissolved in 10 mL of DMF and heated to reflux overnight. The DMF was evaporated and purification of the crude material by flash column chromatography with 5-10% (2M ammonia in methanol) in DCM afforded 1.10 g (48%) of 17c. MS 455 (MH⁺)

Step 17D:

To 120 mg of palladium on charcoal (10 weight %) was added 17c (320 mg, 0.70 mmol) in 7 mL of ethanol. The reaction vessel was purged with hydrogen gas and the reaction was run under 40 psi of hydrogen overnight. The reaction was filtered over Celite and concentrated to give 200 mg (78%) of 17d. MS 365 (MH⁺)

Step 17E:

To 17d (78 mg, 0.21 mmol) and triethylamine (0.044 mL, 0.315 mmol) in 2 mL of DCM cooled to 0° C. was added dropwise trifluoromethanesulfonic anhydride (0.040 mL, 0.235 mmol). After stirring for 1 hour at 0° C., reaction was quenched with 10% sodium bicarbonate solution and extracted with DCM. The DCM layer was washed with brine, dried over MgSO₄, and concentrated to give 100 mg (96%) of 17e as a brown oil. MS 497 (MH⁺)

Step 17F:

To 17e (50 mg, 0.1 mmol) in 1 mL of DMA was added 4-chlorophenylboronic acid (31 mg, 0.2 mmol) and sodium carbonate (32 mg, 0.3 mmol). The mixture was degassed with nitrogen and tetrakis(triphenylphosphine)palladium(0) (6 mg, 5 μmol) was added and the reaction was heated to 85° C. in a sealed tube overnight. After 18 hours, the reaction was cooled to room temperature, filtered, and purified by preparative HPLC to afford 12.5 mg of 17-1 as the trifluoroacetic acid salt. MS 459 (MH⁺)

Using the appropriate starting materials, the following compounds were prepared according to the above procedures.

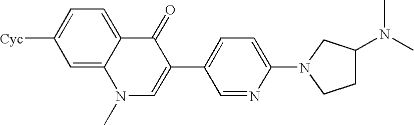

| No. | Cyc | MW | MH⁺ | t_{R} |
|---|---|---|---|---|
| 17-1 | 4-Cl-phenyl | 458.99 | 459.1 | 2.331 |
| 17-2 | 4-CF₃-phenyl | 492.54 | 493.1 | 2.278 |

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A compound having the following structure: 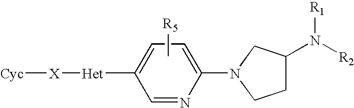
or a stereoisomer, prodrug or pharmaceutically acceptable salt thereof,
wherein:
R₁ is hydrogen, alkyl, substituted alkyl, aryl, substituted aryl, heterocycle, substituted heterocycle, arylalkyl, substituted arylalkyl, heterocyclealkyl or substituted heterocyclealkyl;
R₂ is hydrogen, alkyl, substituted alkyl, —C(O)R₃ or —S(O)₂R₃;
or R₁ and R₂ together with the nitrogen to which they are attached form a heterocycle optionally substituted with 1, 2 or 3 R₄ groups;
R₃ is hydroxy, alkyl, substituted alkyl, —O(alkyl), —O(substituted alkyl), aryl, substituted aryl, heterocycle, substituted heterocycle, arylalkyl, substituted arylalkyl, heterocyclealkyl or substituted heterocyclealkyl;
R₄ is halogen, hydroxy, alkyl, substituted alkyl, —O(alkyl), —O(substituted alkyl), aryl, substituted aryl, heterocycle, substituted heterocycle, arylalkyl, substituted arylalkyl, heterocyclealkyl or substituted heterocyclealkyl;
R⁵ is hydrogen, halogen, cyano, alkyl, substituted alkyl, —O(alkyl), —O(substituted alkyl), —C(O)R₃, —S(O)R₆, —S(O)₂R₃, —C(O)N(R₇)₂, —NHC(O)R₇, or —N(R₇)₂;
R₆ is alkyl, substituted alkyl, —O(alkyl), —O(substituted alkyl), aryl, substituted aryl, heterocycle, substituted heterocycle, arylalkyl, substituted arylalkyl, heterocyclealkyl or substituted heterocyclealkyl;
R₇ is, at each occurrence, the same or different and independently hydrogen, alkyl or substituted alkyl;
Het is 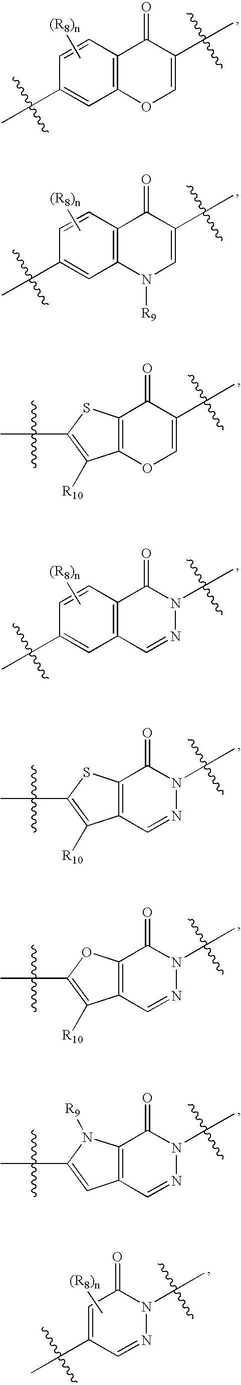 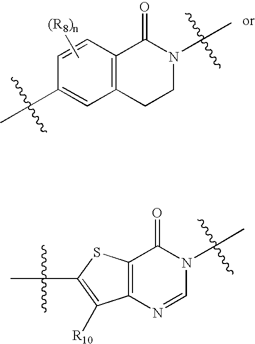
wherein for each Het the connection to the pyridyl ring of structure (I) is from the atom adjacent to the ketone;
R₈ is, at each occurrence, the same or different and independently halogen, alkyl or substituted alkyl;
R₉ is hydrogen, alkyl or substituted alkyl;
R₁₀ is hydrogen, halogen, alkyl or substituted alkyl;
n is 0, 1 or 2;
X is a bond or —O—; and
Cyc is cycloalkyl, substituted cycloalkyl, aryl, substituted aryl, heterocycle or substituted heterocycle.

2. The compound of claim 1 wherein R₁ and R₂ are alkyl.

3. The compound of claim 1 wherein R₁ and R₂ together with the nitrogen to which they are attached form a heterocycle optionally substituted with 1, 2 or 3 R₄ groups.

4. The compound of claim 3 wherein heterocycle is morpholine.

5. The compound of claim 1 wherein R₅ is substituted alkyl.

6. The compound of claim 1 where Het is

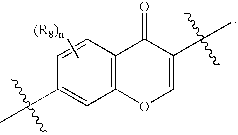

7. The compound of claim 1 where Het is

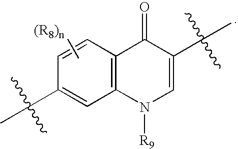

8. The compound of claim 1 where Het is

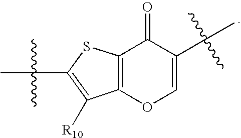

9. The compound of claim 1 where Het is

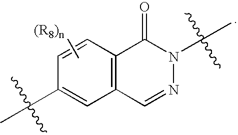

10. The compound of claim 1 where Het is

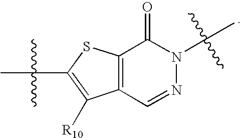

11. The compound of claim 1 where Het is

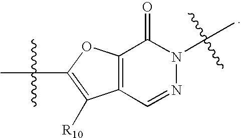

12. The compound of claim 1 where Het is

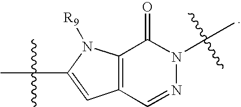

13. The compound of claim 1 where Het is

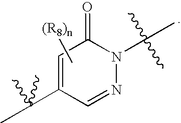

14. The compound of claim 1 where Het is

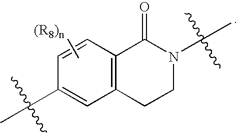

15. The compound of claim 1 where Het is

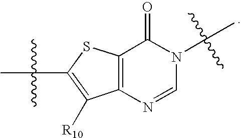

16. The compound of claim 1 wherein X is a bond.

17. The compound of claim 1 wherein Cyc is substituted aryl.

18. A composition comprising a compound of claim 1 in combination with a pharmaceutically acceptable carrier or diluent.

19. A method for antagonizing melanin concentrating hormone in a subject in need thereof comprising administering to the subject an effective amount of a composition of claim 18.

20. A method for treating obesity in a subject in need thereof comprising administering to the subject an effective amount of a composition of claim 18.

21. A method for treating anxiety and/or depression in a subject in need thereof comprising administering to the subject an effective amount of a compound of claim 1 or a composition of claim 18.

22. A method for treating fertility dysfunction in a subject in need thereof comprising administering to the subject an effective amount of a composition of claim 18.

23. A method for treating sexual dysfunction in a subject in need thereof comprising administering to the subject an effective amount of a composition of claim 18.

24. A method for treating urinary disorder in a subject in need thereof comprising administering to the subject an effective amount of a composition of claim 18.

25. A method for treating auto-immune diseases and/or inflammation in a subject in need thereof comprising administering to the subject an effective amount of a composition of claim 18.
